(19) United States
(12) Patent Application Publication
(10) Pub. No.: US 2026/0060971 A1
(43) Pub. Date: Mar. 5, 2026

(54) METHODS OF TREATING SOLID TUMORS HAVING ACTIVATION FGFR3 GENE ALTERATIONS

(71) Applicant: TYRA BIOSCIENCES, INC., Carlsbad, CA (US)

(72) Inventors: Hiroomi TADA, Carlsbad, CA (US); Robert L. HUDKINS, Carlsbad, CA (US); Piyush PATEL, Garnet Valley, PA (US); Daniel C. BENSEN, Carlsbad, CA (US); Timothy Campbell BURN, Sedona, AZ (US)

(21) Appl. No.: 19/106,821

(22) PCT Filed: Aug. 30, 2023
(86) PCT No.: PCT/US2023/073140

§ 371 Date: Feb. 26, 2025

Related U.S. Application Data
(60) Provisional application No. 63/373,933, filed on Aug. 30, 2022.

(51) Int. Cl. A61K 31/444; A61P 35/00

(52) U.S. Cl. CPC A61K 31/444; A61P 35/00

(57) ABSTRACT

The disclosure provides methods of treating cancers that have activating FGFR3 gene alterations by administering a compound of Formula (I) as disclosed herein, or a pharmaceutically acceptable salt of a compound of Formula (I).

### CROSS-REFERENCE TO RELATED APPLICATION

This application claims the benefit of U.S. Provisional Application No. 63/373,933, filed Aug. 30, 2022, the entirety of which is incorporated by reference herein.

### TECHNICAL FIELD

The disclosure is directed to methods of treating cancer using an FGFR3 inhibitor.

### BACKGROUND

The fibroblast growth factor (FGF) family of receptor tyrosine kinases is composed of four highly conserved membrane-associated proteins (FGFR1, FGFR2, FGFR3, and FGFR4) that function to regulate cellular growth, differentiation, and homeostasis. The structures of the FGFRs contain an extracellular domain, transmembrane domain, and an intracellular tyrosine kinase domain. The activity of the intracellular kinase domain is modulated by interactions of the extracellular domain with members of the FGF family of ligands. Twenty-two FGF family members have been identified; of these, eighteen are secreted and act as ligands for the FGFRs. Most of these FGFs act locally, in a paracrine fashion, to affect neighboring cells, while three FGFs (FGF19, FGF21, and FGF23) circulate in the blood and act in an endocrine fashion to regulate FGFRs in distant tissues. When activated, these receptors stimulate multiple cellular pathways including proliferation. See Xie Y, Su N, Yang J, et al. FGF/FGFR signaling in health and disease. Signal Transduct Target Ther. 2020; 5(1):181.

The four FGFRs are structurally homologous but have specific physiologic roles depending on their tissue expression and ligand interactions. The functions of these receptors are often redundant and overlapping, and include roles in embryogenesis, tissue homeostasis, tissue repair, apoptosis, and cellular migration. See Yue S, Li Y, Chen X, et al. FGFR-TKI resistance in cancer: current status and perspectives. J Hematol Oncol. 2021; 14:23. Germline mutations in the receptors can be associated with genetic disorders (e.g., a point mutation in FGFR3 can lead to achondroplasia, the most common form of dwarfism in humans).

In the FGFR family of receptors, oncogenic alterations can occur in the ligand-binding and transmembrane domains, acting as constitutive activators, as well as directly in the intracellular kinase domain. Likewise, in addition to mutations, oncogenesis can occur in the FGFRs through gene rearrangements and gene amplifications. Activating mutations, fusions, or amplifications trigger multiple intracellular pathways, such as the mitogen-activated protein kinase and phosphoinositide 3-kinase pathways, that are common with other oncogenes. See Facchinetti F, Hollebecque A, Bahleda R, et al. Facts and new hopes on selective FGFR inhibitors in solid tumors. Clin Cancer Res. 2020; 26(4):764-74. Using next-generation sequencing (NGS) it has been shown that 7.1% of cancers have some form of FGFR alteration with gene amplification being the most common (66%), followed by activating mutations (26%). The most common cancers manifesting mutations appear to be urothelial (32%), breast (18%), endometrial (13%), lung (13%), and ovarian (9%). See Helsten T, Elkin S, Arthur E, Tomson B N, Carter J, Kurzrock R. The FGFR Landscape in Cancer: Analysis of 4,853 Tumors by Next-Generation Sequencing. Clin Cancer Res. 2016; 22(1):259-67.

Urothelial carcinoma is a major source of morbidity and mortality worldwide with over 420,000 new cases annually and over 165,000 deaths. FGFR3 alterations have been found in up to 80% of non-muscle invasive bladder cancers and up to 15 to 20% of muscle invasive tumors. The most common FGFR3 alteration is a mutation found in the ligand-binding domain, S249C, followed by a mutation in the transmembrane domain, Y375C; these 2 mutations account for approximately 80% of the FGFR3 mutations in bladder cancer.

Current approved and late-stage pan-FGFR inhibitors are susceptible to acquired resistance mutations at the gatekeeper position of the FGFR3 protein. These same pan-FGFR inhibitors are limited by significant toxicity related to inhibiting off-target receptor tyrosine kinases including other isoforms of the FGFR family. This off-target activity limits dosing and theoretically provides further opportunities for acquired resistance to occur due to frequent dose interruptions and dose reductions.

Thus, a need exists for methods of treating cancers that have activating FGFR3 gene alterations using isoform-selective FGFR3 inhibitors.

The compound of Formula (I) is a highly selective inhibitor of FGFR3 that is in development for the treatment of FGFR3-driven cancers. The compound of Formula (I) has the following structure:

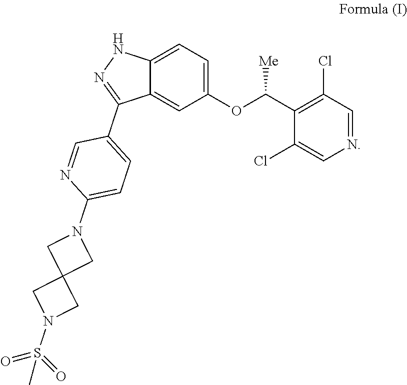 [SMILES: C[C@@H](Oc1ccc2[nH]nc(-c3ccc(N4CC5(C4)CN(S(C)(=O)=O)C5)nc3)c2c1)c1c(Cl)cncc1Cl]

A description of the synthesis of the compound of Formula (I) and its activity, as well as pharmaceutical salts of the compound of Formula (I) and pharmaceutical compositions that may contain the compound of Formula (I) (and/or its salts), can be found in WO2022/147246, which is incorporated by reference herein.

### SUMMARY

The present disclosure meets the need for methods of treating cancers that have activating FGFR3 gene alterations using isoform-selective FGFR3 inhibitors by providing methods of treating cancer in a patient, wherein the methods comprise administering to the patient a compound of Formula (I), or a pharmaceutically acceptable salt thereof. In some embodiments, the cancer has an activating FGFR3 gene alteration.

### BRIEF DESCRIPTION OF THE DRAWINGS

FIG. 1 shows a schematic representation of the study design. Abbreviations: FGFR3=fibroblast growth factor receptor 3, FGFRi=fibroblast growth factor receptor inhibitor, MTD=maximum tolerated dose, mUC=locally advanced/metastatic urothelial carcinoma, RP2D=recommended Phase 2 dose.

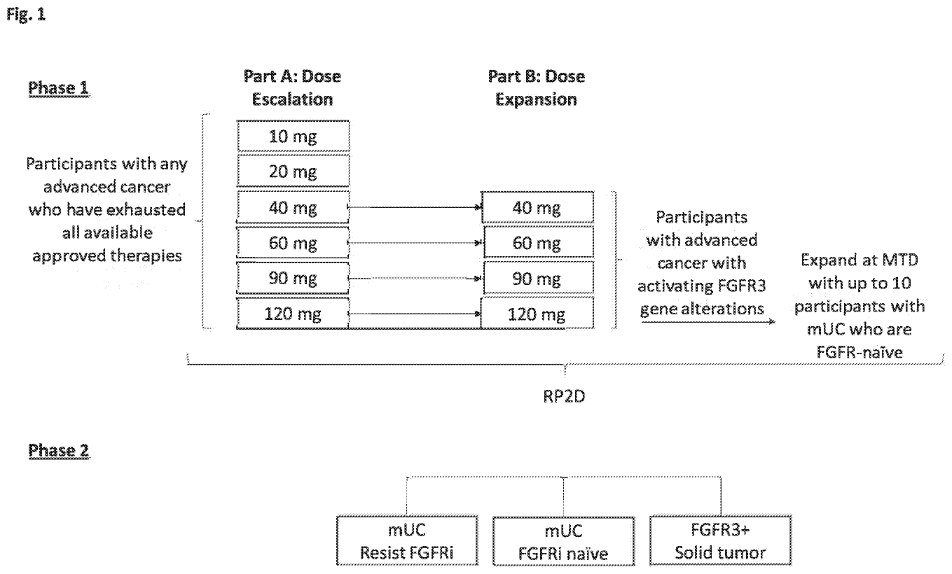

### DETAILED DESCRIPTION OF ILLUSTRATIVE EMBODIMENTS

The disclosure may be more fully appreciated by reference to the following description, including the following definitions and examples. Certain features of the disclosed methods which are described herein in the context of separate aspects, may also be provided in combination in a single aspect. Alternatively, various features of the disclosed methods that are, for brevity, described in the context of a single aspect, may also be provided separately or in any subcombination.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

The term “pharmaceutically acceptable salt” refers to a salt of a compound that does not cause significant irritation to an organism to which it is administered and does not abrogate the biological activity and properties of the compound. In several embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, benzenesulfonic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine. Particularly preferred salts of the compound of Formula (I) are described in WO2022/147246.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. It is understood that, in any compound described herein having one or more chiral centers, all possible diastereomers are also envisioned. It is understood that, in any compound described herein all tautomers are envisioned. It is also understood that, in any compound described herein, all isotopes of the included atoms are envisioned. For example, any instance of hydrogen, may include hydrogen-1 (protium), hydrogen-2 (deuterium), hydrogen-3 (tritium) or other isotopes; any instance of carbon may include carbon-12, carbon-13, carbon-14, or other isotopes; any instance of oxygen may include oxygen-16, oxygen-17, oxygen-18, or other isotopes; any instance of fluorine may include one or more of fluorine-18, fluorine-19, or other isotopes; any instance of sulfur may include one or more of sulfur-32, sulfur-34, sulfur-35, sulfur-36, or other isotopes.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a compound of Formula (I) and that is compatible therewith.

As used herein, “subject,” “host,” “patient,” “participant,” and “individual” are used interchangeably and shall be given its ordinary meaning and shall also refer to an organism that has FGFR proteins. This includes mammals, e.g., a human, a non-human primate, ungulates, canines, felines, equines, mice, rats, and the like. The term “mammal” includes both human and non-human mammals.

The terms “treatment,” “treating,” “treat” and the like shall be given its ordinary meaning and shall also include herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein shall be given its ordinary meaning and shall also cover any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; and/or (c) relieving the disease symptom, e.g., causing regression of the disease or symptom.

The term “administering,” when used in the context of administering a therapeutic agent to a patient, refers to introducing the therapeutic agent into the patient's body. For example, therapeutic agents may be introduced into a patient's body orally, nasally, subcutaneously, intravenously, intravesically, intramuscularly, transdermally, vaginally, rectally or in any combination thereof.

The terms “cancer,” “neoplasm,” and “tumor” are used interchangeably herein, shall be given its ordinary meaning and shall also refer to cells which exhibit relatively autonomous growth, so that they exhibit an aberrant growth phenotype characterized by a significant loss of control of cell proliferation. In general, cells of interest for detection or treatment in the present application include precursors, precancerous (e.g., benign), malignant, pre-metastatic, metastatic, and non-metastatic cells.

As used herein, “an activating FGFR3 gene alteration” refers to a mutation or rearrangement of the FGFR3 gene, relative to the wild-type FGFR3 gene, such that the FGFR3 gene having an activating FGFR3 gene alteration encodes an FGFR kinase that has greater FGFR3 kinase activity that the FGFR kinase that is encoded by the wild-type FGFR3 gene.

In some aspects, the disclosure is directed to methods of treating cancer in a patient, wherein the method comprises administering to the patient a compound of Formula (I), or a pharmaceutically acceptable salt thereof.

In some aspects, the disclosure is directed to methods of treating cancer in a patient, wherein the method comprises administering to the patient a compound of Formula (I), or a pharmaceutically acceptable salt thereof, and wherein the cancer has an activating FGFR3 gene alteration.

In some embodiments, the cancer is urothelial cancer, breast cancer, endometrial cancer, lung cancer, ovarian cancer, or bladder cancer.

In some embodiments, the cancer is urothelial cancer.

In other embodiments, the cancer is urothelial carcinoma.

In other me embodiments, the cancer is breast cancer.

In other embodiments, the cancer is endometrial cancer.

In other embodiments, the cancer is lung cancer.

In other embodiments, the cancer is ovarian cancer.

In other embodiments, the cancer is bladder cancer.

In some embodiments, the cancer is non-muscle invasive bladder cancer (NMIBC).

In other embodiments, the cancer is a locally advanced solid tumor.

In other embodiments, the cancer is a metastatic solid tumor.

In some aspects of the disclosed methods, the cancer has an activating FGFR3 gene alteration.

In some embodiments, the activating FGFR3 gene alteration is a mutation. As used in this context, the term “mutation” refers to a change in the FGFR3 gene that results in the encoded FGFR3 kinase having a different amino acid sequence that the wild-type FGFR3 kinase. Methods of identifying FGFR3 mutations are known in the art.

In some embodiments, the mutation is any one or more of the following:
FGFR3 p.S84L;
FGFR3 p.G380R;
FGFR3 p.R621H;
FGFR3 p.R248C;
FGFR3 p.G380E;
FGFR3 p.K650E;
FGFR3 p.S249C;
FGFR3 p.A391V;
FGFR3 p.K650M;
FGFR3 p.P250R;
FGFR3 p.A391E;
FGFR3 p.K650T;
FGFR3 p.T264M;
FGFR3 p.M528I;
FGFR3 p.K650N;
FGFR3 p.G370C;
FGFR3 p.N540D;
FGFR3 p.R669Q;
FGFR3 p.S371C;
FGFR3 p.N540S;
FGFR3 p.G697C;
FGFR3 p.Y373C; or
FGFR3 p.N540K.

In other embodiments, the mutation is any one or more of the following:
FGFR3 p.V553M;
FGFR3 p.V555M; or
FGFR3 p.V555L.

In other embodiments, the activating FGFR3 gene alteration is a fusion gene mutation. As used in this context, a “fusion” is a gene that results from the joining of two previously independent genes.

In some embodiments, the fusion is an FGFR3 rearrangement with an intact FGFR3 kinase domain and:
Breakpoint in intron 17 or exon 18 of FGFR3 and a known partner gene (e.g., TACC3, BAIAP2L1);
Breakpoint in intron 17 or exon 18 of FGFR3 and an in-frame novel partner gene; or
Breakpoint in intron 17 or exon 18 of FGFR3 and an intra-genic region or out-of-frame partner gene.

In some aspects of the methods of the disclosure, the patient is administered a compound of Formula (I).

In other aspects of the methods of the disclosure, the patient is administered a pharmaceutically acceptable salt of a compound of Formula (I).

In some embodiments, the pharmaceutically acceptable salt of a compound of Formula (I) is the hydrochloride salt, besylate salt, maleate salt, tosylate salt, sulfate salt, 2-hydroxyethanesulfonate salt, ethanesulfonate (esylate) salt, mesylate salt, di-mesylate salt, R-camsylate salt, S-camsylate salt, or hydrobromide salt.

In some embodiments of the disclosed methods, the patient is administered the compound of Formula (I), or a pharmaceutically acceptable salt of a compound of Formula (I) (on a Formula (I) basis), in an amount of 10 mg-120 mg per day, such as, for example, one of 10 mg/day, 15 mg/day, 20 mg/day, 25 mg/day, 30 mg/day, 35 mg/day, 40 mg/day, 45 mg/day, 50 mg/day, 55 mg/day, 60 mg/day, 65 mg/day, 70 mg/day, 75 mg/day, 80 mg/day, 85 mg/day, 90 mg/day, 95 mg/day, 100 mg/day, 105 mg/day, 110 mg/day, 115 mg/day, or 120 mg/day. In other embodiments of the disclosed methods, the patient is administered the compound of Formula (I), or a pharmaceutically acceptable salt of a compound of Formula (I) (on a Formula (I) basis), in an amount of 10 mg-200 mg per day, such as, for example, one of 10 mg/day, 15 mg/day, 20 mg/day, 25 mg/day, 30 mg/day, 35 mg/day, 40 mg/day, 45 mg/day, 50 mg/day, 55 mg/day, 60 mg/day, 65 mg/day, 70 mg/day, 75 mg/day, 80 mg/day, 85 mg/day, 90 mg/day, 95 mg/day, 100 mg/day, 105 mg/day, 110 mg/day, 115 mg/day, 120 mg/day, 125 mg/day, 130 mg/day, 135 mg/day, 140 mg/day, 145 mg/day, 150 mg/day, 155 mg/day, 160 mg/day, 165 mg/day, 170 mg/day, 175 mg/day, 180 mg/day, 185 mg/day, 190 mg/day, 195 mg/day, or 200 mg/day. When the compound of Formula (I) is administered as a pharmaceutically acceptable salt, the amount of the salt that is administered is based on the compound of Formula (I). That is, the amount of the salt that is administered is an amount that contains the specified amount of Formula (I) free base.

In some embodiments of the disclosed methods, the patient is administered the compound of Formula (I), or a pharmaceutically acceptable salt of a compound of Formula (I) (on a Formula (I) basis), in an amount of 10 mg per day.

In some embodiments of the disclosed methods, the patient is administered the compound of Formula (I), or a pharmaceutically acceptable salt of a compound of Formula (I) (on a Formula (I) basis), in an amount of 20 mg per day.

In some embodiments of the disclosed methods, the patient is administered the compound of Formula (I), or a pharmaceutically acceptable salt of a compound of Formula (I) (on a Formula (I) basis), in an amount of 40 mg per day.

In some embodiments of the disclosed methods, the patient is administered the compound of Formula (I), or a pharmaceutically acceptable salt of a compound of Formula (I) (on a Formula (I) basis), in an amount of 60 mg per day.

In some embodiments of the disclosed methods, the patient is administered the compound of Formula (I), or a pharmaceutically acceptable salt of a compound of Formula (I) (on a Formula (I) basis), in an amount of 75 mg per day.

In some embodiments of the disclosed methods, the patient is administered the compound of Formula (I), or a pharmaceutically acceptable salt of a compound of Formula (I) (on a Formula (I) basis), in an amount of 80 mg per day.

In some embodiments of the disclosed methods, the patient is administered the compound of Formula (I), or a pharmaceutically acceptable salt of a compound of Formula (I) (on a Formula (I) basis), in an amount of 90 mg per day.

In some embodiments of the disclosed methods, the patient is administered the compound of Formula (I), or a pharmaceutically acceptable salt of a compound of Formula (I) (on a Formula (I) basis), in an amount of 120 mg per day.

In some embodiments of the methods of the disclosure, the daily amount of the compound of Formula (I), or a pharmaceutically acceptable salt of a compound of Formula (I) (on a Formula (I) basis), is administered in a single dose.

In some embodiments of the methods of the disclosure, the daily amount of the compound of Formula (I), or a pharmaceutically acceptable salt of a compound of Formula (I) (on a Formula (I) basis), is given in a multiple doses, wherein each of the multiple doses contains a portion of the daily amount.

In some embodiments, the daily amount of the compound of Formula (I), or a pharmaceutically acceptable salt of a compound of Formula (I) (on a Formula (I) basis), is given in two doses, wherein each of the two doses contains a portion of the daily amount.

In some embodiments, the daily amount of the compound of Formula (I), or a pharmaceutically acceptable salt of a compound of Formula (I) (on a Formula (I) basis), is given in two doses, wherein each of the two doses contains one half of the daily amount.

In the methods of the disclosure, the compound of Formula (I), or pharmaceutically acceptable salt of a compound of Formula (I), may be administered by any suitable route of administration, such as, for example, orally, nasally, subcutaneously, intravenously, intravesically, intramuscularly, transdermally, vaginally, rectally or in any combination thereof.

In some embodiments, the compound of Formula (I), or pharmaceutically acceptable salt of a compound of Formula (I), is administered orally.

In the methods of the disclosure, the compound of Formula (I), or pharmaceutically acceptable salt of a compound of Formula (I), may be administered in any suitable pharmaceutical dosage form. Suitable dosage forms include, but are not limited to capsules, tablets, powders, suspensions, solutions, and the like. The pharmaceutical dosage form is typically formulated to provide a therapeutically effective amount of a compound of Formula (I), or pharmaceutically acceptable salt of a compound of Formula (I), as the active ingredient. In some embodiments, the pharmaceutical dosage form also contains one or more pharmaceutically acceptable excipients.

In some embodiments of the disclosure, the compound of Formula (I), or pharmaceutically acceptable salt of a compound of Formula (I), is administered for at least 28 days. In some aspects, the administration is at least once daily. In some aspects, the administration is once daily.

In other embodiments of the disclosure, the compound of Formula (I), or pharmaceutically acceptable salt of a compound of Formula (I), is administered for 28 days. In some aspects, the administration is at least once daily. In some aspects, the administration is once daily.

In some aspects, the disclosed methods comprise administering the compound of Formula (I), or pharmaceutically acceptable salt of a compound of Formula (I), together with additional therapy.

In some embodiments, the additional therapy includes one or more of radiation therapy, chemotherapy, surgery (e.g., at least partial resection of the tumor).

In some embodiments, the additional therapy is chemotherapy, i.e., administering one or more additional therapeutic agents.

In some embodiments, the additional therapeutic agent is a checkpoint inhibitor.

In some embodiments, the checkpoint inhibitor is a PD-1/PD-L1 inhibitor, such as, for example, one or more of pembrolizumab, nivolumab, avelumab, durvalumab, atezolizumab, cemiplimab, dostarlimab, JTX-4014, spartalizumab, camrelizumab, sintilimab, tislelizumab, toripalimab, INCMGA00012 (MGA012), AMP-224, or AMP-514 (MEDI0680).

In other embodiments, the checkpoint inhibitor is a CTLA-4 inhibitor, such as, for example, one or more of ipilimumab, tremelimumab, or AGEN-1884.

In some embodiments, the additional therapeutic agent is an antibody-drug conjugate, such as, for example, enfortumab vendotin, sacituzumab govitecan, disitamab vedotin, or Vic-trastuzumab duocarmazine (SYD985).

In other embodiments, the additional therapeutic agent is cisplatin, carboplatin, gemcitabine, docetaxel, paclitaxel, vinflunine, methotrexate, vinblastine, mitomycin, valrubicin, or doxorubicin.

In some embodiments, the additional therapeutic agent is an MEK inhibitor, such as, for example, trametinib, cobimetinib, or binimetinib.

In some embodiments, the additional therapeutic agent is a PARP inhibitor, such as, for example, olaparib, veliparib, niraparib, rucaparib, or talazoparib.

In some embodiments, the additional therapeutic agent is an HER2 inhibitor, such as, for example, lapatinib, afatinib, AZD8931, AST-1306, AEE-788, canertinib (CI-1033), CP724, CP714, CUDC-101, TAK-285, AC-480 (BMS-599626), dacomitinib (PF299804 PF299) (Dacomitinib), or pelitinib (EKB-569).

In some embodiments, the additional therapeutic agent is an SHP2 inhibitor, such as, for example, TNO-155, or RMC-4630.

In some embodiments, the additional therapeutic agent is an antibody.

In some embodiments, the antibody is an HER2 antibody such as one or more of trastuzumab or pertuzumab.

In some embodiments, the antibody is a bispecific antibodies such as one or more of MM-111 or ertumaxomab.

In some embodiments, the additional therapy is a biologic immunotherapy such as, for example, intravesicle BCG (Bacillus Calmette-Guerin).

In embodiments of the disclosed methods that comprise administering the compound of Formula (I), or pharmaceutically acceptable salt of a compound of Formula (I), together with additional therapy, the compound of Formula (I) (or salt thereof) is administered before, during, or after the administration or application of the additional therapy.

In embodiments of the disclosed methods wherein the additional therapy is a chemotherapy, the chemotherapeutic agent may be administered by any suitable route of administration, such as, for example, orally, nasally, subcutaneously, intravenously, intravescically, intramuscularly, transdermally, vaginally, rectally, or in any combination thereof.

In some embodiments, the compound of Formula (I) (or salt thereof) is administered before administration or application of the additional therapy.

In some embodiments, the compound of Formula (I) (or salt thereof) is administered during administration or application of the additional therapy.

In some embodiments, the compound of Formula (I) (or salt thereof) is administered after administration or application of the additional therapy.

In some aspects of the methods of the disclosure, the cancer exhibits a complete response (CR) or a partial response (PR), as evaluated by the Response Evaluation Criteria in Solid Tumors (RECIST) v1.1 criteria, to the administration of the compound of Formula (I), or pharmaceutically acceptable salt of a compound of Formula (I).

In some embodiments, the cancer exhibits a complete response (CR), as evaluated by the Response Evaluation Criteria in Solid Tumors (RECIST) v1.1 criteria, to the administration of the compound of Formula (I), or pharmaceutically acceptable salt of a compound of Formula (I).

In other embodiments, the cancer exhibits a partial response (PR), as evaluated by the Response Evaluation Criteria in Solid Tumors (RECIST) v1.1 criteria, to the administration of the compound of Formula (I), or pharmaceutically acceptable salt of a compound of Formula (I).

The disclosure is also directed to the following aspects:

Aspect 1. A method of treating cancer in a patient in need thereof, comprising administering to the patient a compound of Formula (I),

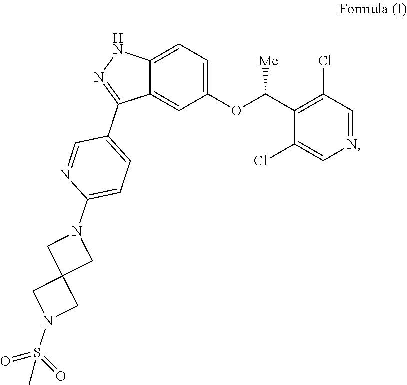 [SMILES: C[C@@H](Oc1ccc2[nH]nc(-c3ccc(N4CC5(C4)CN(S(C)(=O)=O)C5)nc3)c2c1)c1c(Cl)cncc1Cl]

or a pharmaceutically acceptable salt thereof.

Aspect 2. The method of aspect 1, wherein the cancer has an activating FGFR3 gene alteration.

Aspect 3. The method of aspect 1 or aspect 2, wherein the cancer is urothelial cancer, breast cancer, endometrial cancer, lung cancer, ovarian cancer, or bladder cancer.

Aspect 4. The method of aspect 3, wherein the cancer is urothelial cancer.

Aspect 5. The method of aspect 3, wherein the cancer is urothelial carcinoma.

Aspect 6. The method of aspect 3, wherein the cancer is breast cancer.

Aspect 7. The method of aspect 3, wherein the cancer is endometrial cancer.

Aspect 8. The method of aspect 3, wherein the cancer is lung cancer.

Aspect 9. The method of aspect 3, wherein the cancer is ovarian cancer.

Aspect 10. The method of aspect 3, wherein the cancer is bladder cancer.

Aspect 11. The method of any one of aspects 1 to 10, wherein the cancer is a locally advanced solid tumor.

Aspect 12. The method of any one of aspects 1 to 11, wherein the cancer is a metastatic solid tumor.

Aspect 13. The method of any one of aspects 2 to 12, wherein the activating FGFR3 gene alteration is a mutation.

Aspect 14. The method of aspect 13, wherein the mutation is or comprises one or more of
FGFR3 p.S84L;
FGFR3 p.G380R;
FGFR3 p.R621H;
FGFR3 p.R248C;
FGFR3 p.G380E;
FGFR3 p.K650E;
FGFR3 p.S249C;
FGFR3 p.A391V;
FGFR3 p.K650M;
FGFR3 p.P250R;
FGFR3 p.A391E;
FGFR3 p.K650T;
FGFR3 p.T264M;
FGFR3 p.M528I;
FGFR3 p.K650N;
FGFR3 p.G370C;
FGFR3 p.N540D;
FGFR3 p.R669Q;
FGFR3 p.S371C;
FGFR3 p.N540S;
FGFR3 p.G697C;
FGFR3 p.Y373C; or
FGFR3 p.N540K.

Aspect 15. The method of aspect 13, wherein the mutation is or comprises one or more of
FGFR3 p.V553M;
FGFR3 p.V555M; or
FGFR3 p.V555L.

Aspect 16. The method of any one of aspects 2 to 12, wherein the activating FGFR3 gene alteration is a fusion.

Aspect 17. The method of aspect 16, wherein the fusion is an FGFR3 rearrangements with an intact FGFR3 kinase domain and:
Breakpoint in intron 17 or exon 18 of FGFR3 and a known partner gene (e.g., TACC3, BAIAP2L1);
Breakpoint in intron 17 or exon 18 of FGFR3 and an in-frame novel partner gene; or
Breakpoint in intron 17 or exon 18 of FGFR3 and an intra-genic region or out-of-frame partner gene.

Aspect 18. The method of any one of the preceding aspects, wherein the patient is administered a compound of Formula (I).

Aspect 19. The method of any one of aspects 1 to 17, wherein the patient is administered a pharmaceutically acceptable salt of a compound of Formula (I).

Aspect 20. The method of aspect 19, wherein the pharmaceutically acceptable salt of a compound of Formula (I) is the besylate salt.

Aspect 21. The method of any one of the preceding aspects, wherein the patient is administered the compound of Formula (I), or a pharmaceutically acceptable salt of a compound of Formula (I) (on a Formula (I) basis), in an amount of 10 mg-120 mg per day of.

Aspect 22. The method of aspect 21, wherein the patient is administered the compound of Formula (I), or a pharmaceutically acceptable salt of a compound of Formula (I) (on a Formula (I) basis), in an amount of 10 mg per day.

Aspect 23. The method of aspect 21, wherein the patient is administered the compound of Formula (I), or a pharmaceutically acceptable salt of a compound of Formula (I) (on a Formula (I) basis), in an amount of 20 mg per day.

Aspect 24. The method of aspect 21, wherein the patient is administered the compound of Formula (I), or a pharmaceutically acceptable salt of a compound of Formula (I) (on a Formula (I) basis), in an amount of 40 mg per day.

Aspect 25. The method of aspect 21, wherein the patient is administered the compound of Formula (I), or a pharmaceutically acceptable salt of a compound of Formula (I) (on a Formula (I) basis), in an amount of 60 mg per day.

Aspect 26. The method of aspect 21, wherein the patient is administered the compound of Formula (I), or a pharmaceutically acceptable salt of a compound of Formula (I) (on a Formula (I) basis), in an amount of 90 mg per day.

Aspect 27. The method of aspect 21, wherein the patient is administered the compound of Formula (I), or a pharmaceutically acceptable salt of a compound of Formula (I) (on a Formula (I) basis), in an amount of 120 mg per day.

Aspect 28. The method of any one of aspects 21-27, wherein the compound of Formula (I), or pharmaceutically acceptable salt of a compound of Formula (I), is administered orally.

Aspect 29. The method of any one of the preceding aspects, wherein the compound of Formula (I), or pharmaceutically acceptable salt of a compound of Formula (I), is administered for at least 28 days.

Aspect 30. The method of aspect 29, wherein the compound of Formula (I), or pharmaceutically acceptable salt of a compound of Formula (I), is administered for 28 days.

Aspect 31. The method of any one of aspects 1 to 30, wherein the cancer exhibits a complete response (CR) or a partial response (PR), as evaluated by the Response Evaluation Criteria in Solid Tumors (RECIST) v1.1 criteria, to the administration of the compound of Formula (I), or pharmaceutically acceptable salt of a compound of Formula (I).

Aspect 32. The method of aspect 31, wherein the cancer exhibits a complete response (CR), as evaluated by the Response Evaluation Criteria in Solid Tumors (RECIST) v1.1 criteria, to the administration of the compound of Formula (I), or pharmaceutically acceptable salt of a compound of Formula (I).

Aspect 33. The method of aspect 31 or aspect 32, wherein the cancer exhibits a partial response (PR), as evaluated by the Response Evaluation Criteria in Solid Tumors (RECIST) v1.1 criteria, to the administration of the compound of Formula (I), or pharmaceutically acceptable salt of a compound of Formula (I).

The Example provided below further illustrates and exemplifies the disclosed methods. It is to be understood that the scope of the present invention is not limited in any way by the scope of the following Example.

### EXAMPLE

| Abbreviation | Definition |
|---|---|
| AE | adverse event |
| ALT | alanine aminotransferase |
| ANC | absolute neutrophil count |
| AST | aspartate aminotransferase |
| AUC | area under the concentration-time curve |
| AUC_{0-∞} | area under the concentration time-curve from time zero to infinity |
| AUC₀₋ₗₐₛₜ | area under the concentration-time curve from time zero to the time of |
| | the last quantifiable concentration |
| AUC_{Tau} | area under plasma concentration-time curve over dosing interval |
| BOR | best overall response |
| CDx | companion diagnostic |
| CI | confidence interval |
| CLIA | Clinical Laboratory Improvement Amendments |
| CL/F | apparent oral clearance |
| CSR | serum Collagen Type X |
| Cₘₐₓ | maximum concentration |
| Cₘᵢₙ | minimum concentration |
| COVID-19 | coronavirus disease 2019 |
| CR | complete response |
| CSR | central serous retinopathy |
| CT | computed tomography |
| CTA | clinical trial assay |
| CTCAE | Common Terminology Criteria for Adverse Events |
| ctDNA | circulating tumor DNA |
| CV | coefficient of variation |
| CYP | cytochrome p450 |
| d | day(s) |
| DCR | disease control rate |
| DDI | drug-drug interaction |
| DILI | drug-induced liver injury |
| DL | dose level |
| DLT | dose-limiting toxicity |
| DOR | duration of response |
| DSMB | Data and Safety Monitoring Board |
| EAS1 | Phase 1 Efficacy-evaluable Analysis Set |
| EAS2 | Phase 2 Efficacy-evaluable Analysis Set |
| EC | Ethics Committee |
| ECG | electrocardiogram |
| ECOG PS | Eastern Cooperative Oncology Group Performance Status |
| eCRF | electronic Case Report Form |
| EDC | electronic data capture |
| EIU | exposure in utero |
| EOT | End of Treatment |
| ESMO | European Society for Medical Oncology |
| FDA | Food and Drug Administration |
| FDG | fluorodeoxyglucose |
| FFPE | formalin-fixed paraffin-embedded |
| FGF | fibroblast growth factor |
| FGFR | fibroblast growth factor receptor |
| FSH | follicle stimulating hormone |
| GCP | Good Clinical Practice |
| GFR | glomerular filtration rate |
| h | hour(s) |
| HbA1c | glycated hemoglobin |
| HED | human equivalent dose |
| HIV | human immunodeficiency virus |
| HNSTD | highest non-severely toxic dose |
| HRT | hormone replacement therapy |
| i3 + 3 | interval 3 + 3 |
| IB | Investigator's Brochure |
| ICF | Informed Consent Form |
| ICH | International Council for Harmonisation of Technical Requirements |
| | for Pharmaceuticals for Human Use |
| ID | identification |
| IND | investigational new drug |
| INR | international normalized ratio |
| IRB | Institutional Review oard |
| IRT | Interactive Response Technology |
| IV | intravenous |
| KPS | Karnofsky Performance Status |
| LFT | liver function test |
| NGS | next-generation sequencing |
| MATE | multidrug and toxin extrusion |
| MedDRA | Medical Dictionary for Regulatory Activities |
| MRI | magnetic resonance imaging |
| MTD | maximum tolerated dose |
| ORR | objective response rate |
| OS | overall survival |
| PCR | polymerase chain reaction |
| PD | progressive disease |
| PET | positron emission tomography |
| PFS | progression-free survival |
| PI | Principal Investigator |
| PK | pharmacokinetic(s) |
| PR | partial response |
| QRS | duration of ventricular depolarization and contraction interval |
| QT | interval between Q and T wave |
| QTcF | standard interval between Q and T wave corrected for heart rate using |
| | Fridericia's formula |
| RECIST v1.1 | Response Evaluation Criteria in Solid Tumors version 1.1 |
| RP2D | recommended Phase 2 dose |
| RVO | retinal vascular occlusion |
| SAE | serious adverse event |
| SAP | Statistical Analysis Plan |
| SARS-CoV-2 | severe acute respiratory syndrome coronavirus 2 |
| SD | stable disease |
| SoA | schedule of assessments |
| SAS | Safety Analysis Set |
| SOP | standard operating procedure |
| SRC | Safety Review Committee |
| STD | severely toxic dose |
| t_{1/2} | apparent terminal half-life |
| TKI | tyrosine kinase inhibitor |
| Tₘₐₓ | time to reach Cₘₐₓ |
| TRAE | treatment-related adverse event |
| TTR | time to response |
| ULN | upper limit of normal |
| US | United States |
| USM | urgent safety measure |
| Vd/F | apparent volume of distribution |

Study Title: A Multicenter, Open-label Phase 1/2 Study of Formula (I) in Advanced Urothelial Carcinoma and Other Solid Tumors with Activating FGFR3 Gene Alterations
Objectives: Primary Objectives:
   To determine the optimal and maximum tolerated doses (MTD), and the recommended Phase 2 dose (RP2D) of Formula (I) in participants with advanced solid tumors (Phase 1, Parts A and B)
   To evaluate the preliminary antitumor activity of Formula (I) at the RP2D in participants in selected tumor expansion cohorts with activating fibroblast growth factor receptor (FGFR) 3 gene alterations (Phase 2)
   Secondary Objectives:
   To characterize the safety and tolerability of Formula (I) in participants with advanced cancer (Phase 1, Parts A and B, and Phase 2)
   To conduct a preliminary characterization of the pharmacokinetics (PK) and pharmacodynamics in participants treated with Formula (I) (Phase 1, Parts A and B, and Phase 2)
   To characterize objective response rate (ORR) in participants with activating FGFR3 gene alterations (Phase 1, Part B)
   To characterize duration of response (DOR; Phase 1, Part B and Phase 2)
   To characterize disease control rate (DCR)>12 weeks (Phase 1, Part B and Phase 2)
   To characterize time to response (TTR; Phase 1, Part B and Phase 2)
   To characterize progression-free survival (PFS) in participants treated with Formula (I) in specific tumor expansion cohorts (Cohorts 1 and 2 of Phase 2 only)
   Exploratory Objectives:
   To identify potential biomarkers of FGFR engagement/inhibition, toxicity, tumor response, and resistance to Formula (I) in blood, urine, and tissues
   To evaluate concordance between tissue and confirmatory clinical trial assay (CTA) results for the detection of FGFR3 gene alterations in participants with locally advanced/metastatic urothelial carcinoma
   Estimate overall survival (OS) in Cohorts 1 and 2 of Phase 2

### Study Design:

This is a Phase 1/2, open-label, international, multicenter, dose escalation/dose expansion study of Formula (I) that is implemented in several parts.

### Phase 1, Part A: Dose Escalation

Participants with any advanced solid tumor for which there are no approved standard therapies are eligible to be enrolled, regardless of FGFR3 mutation status. The Formula (I) starting dose in Part A is 10 mg daily (DL 1), with dose escalation schema as follows:
DL1: 10 mg daily
DL2: 20 mg daily
DL3: 40 mg daily
DL4: 60 mg daily
DL5: 90 mg daily
DL6: 120 mg daily

The study uses an i3+3 design with a target toxicity rate of the MTD of 0.3 (0.25 to 0.35). Each dose level (DL) is assessed through the dose-limiting toxicity (DLT) evaluation period (Cycle 1 [28 days]) before the next DL is enrolled. Depending on the number of participants and the number of DLTs observed, the decision is to escalate to a higher dose or, if at DL6, continue enrollment in the highest dose (E), stay at the same dose (S), de-escalate to the previous lower dose (D), or de-escalate to the previous lower dose and never repeat current dose (DU).

DL1 and DL2 proceed in an accelerated fashion, monitoring toxicity in 1 participant during the DLT evaluation period. Any ≥Grade 2 treatment-related adverse event (TRAE) constitutes a DLT for DL1 and DL2. If no ≥Grade 2 TRAE occurs at DL1, the next participant is enrolled to DL2. Similarly, if no ≥Grade 2 TRAE occurs at DL2, the next cohort dose escalates (ie, enrolled to DL3). If either DL1 or DL2 enroll additional participants due to this stricter DLT definition, the study converts to an i3+3 design and all subsequent dose escalation decisions use the same DLT definitions as for DL3 and above (ie, no longer use ≥Grade 2 TRAEs as the definition for DLTs).

DL3 and above enroll a minimum of 3 participants per DL. These cohorts may enroll 1 to 3 additional participants at a time if required by the DLT rules. Dose escalation continues up to DL6, until the MTD is determined, or until approximately 30 participants have been enrolled in Part A.

### Phase 1, Part B: Dose Expansion

When DL3 has cleared dose escalation, dose expansion cohorts begin enrolling in parallel to further explore pharmacodynamics and biomarkers in FGFR3-mutated cancers. Enrollment is limited to participants with advanced/metastatic solid tumors with an FGFR3 activating gene alteration for which an FGFR-directed therapy is appropriate. Participants in dose expansion undergo additional analyses for biomarkers and pharmacodynamic endpoints including, but not limited to, on treatment and post-progression assessment of ctDNA, serial blood biomarker analyses, and skin biopsies to assess FGFR target inhibition.

Eligible FGFR3 gene mutations and fusions are defined as follows:

Eligible FGFR3 gene mutations are listed in the table below.

Known and/or Likely Pathogenic Activating Fibroblast Growth
Factor Receptor 3 Gene Mutations
Gene Mutation

| | | |
|---|---|---|
| FGFR3 p.S84L | FGFR3 p.G380R | FGFR3 p.R621H |
| FGFR3 p.R248C | FGFR3 p.G380E | FGFR3 p.K650E |
| FGFR3 p.S249C | FGFR3 p.A391V | FGFR3 p.K650M |
| FGFR3 p.P250R | FGFR3 p.A391E | FGFR3 p.K650T |
| FGFR3 p.T264M | FGFR3 p.M528I | FGFR3 p.K650N |
| FGFR3 p.G370C | FGFR3 p.N540D | FGFR3 p.R669Q |
| FGFR3 p.S371C | FGFR3 p.N540S | FGFR3 p.G697C |
| FGFR3 p.Y373C | FGFR3 p.N540K | |

### Eligible Fibroblast Growth Factor Receptor 3 Fusions

FGFR3 rearrangements with an intact FGFR3 kinase domain and:
Breakpoint in intron 17 or exon 18 of FGFR3 and a known partner gene (eg, TACC3, BAIAP2L1).
Breakpoint in intron 17 or exon 18 of FGFR3 and an in-frame novel partner gene.
Breakpoint in intron 17 or exon 18 of FGFR3 and an intra-genic region or out-of-frame partner gene.

Secondary Fibroblast Growth Factor Receptor 3 Mutations Responsible for Acquired Resistance to Current Generation Fibroblast Growth Factor Receptor Inhibitors:
FGFR3 p.V553M
FGFR3 p.V555M
FGFR3 p.V555L

Part B also utilizes the i3+3 design, but dose escalation does not apply. The cohort can continue expansion based on the toxicity probability resulting from participants observed in both Parts A and B at that DL. Because Part A is dose-escalating to DL4 through DL6 in parallel, DLTs observed in Part B may result in a need to pause or stop enrollment into Part A. Depending on the number of participants and number of DLTs observed, the decision is to continue Part B expansion (E), continue Part B expansion but pause enrollment in Part A (S), or stop expansion to the DL and discontinue Part A (D or DU). Cohort expansion continues if the toxicity probability is not above 0.35 for the entire DL that includes data from both Parts A and B. If Part B results in a pause to enrollment in Part A, enrollment to higher doses in Part A proceeds again if dose expansion and continued monitoring of Part B reduces the toxicity rate to less than 0.25.

Up to 10 additional participants with locally advanced/metastatic FGFR3 mutation positive urothelial carcinoma who have not received a prior FGFR inhibitor are enrolled at the MTD in Part B when the MTD is determined in Part A. This cohort evaluates the PK of a tablet formulation that is used in the Phase 2 portion of the study. PK and AEs are reviewed by the SRC after 3 to 5 participants to ensure there are no safety concerns before completing enrolment of this expansion cohort.

### Intra-Participant Dose Escalation Decisions—Phase 1, Part A and B

Intra-participant dose escalation are allowed for participants in these cohorts under the following circumstances:
A higher dose has been cleared in dose escalation (Part A) and was deemed to be tolerated.
The participant has not had a >Grade 1 TRAE while taking Formula (I).
If a ≥Grade 3 treatment-related toxicity in any participant occurs at a given DL, intra-participant dose escalation is not allowed at that DL.
A higher dose under study in an enrolling or completed cohort may be safe and potentially more effective in treating the participant's tumor than the current dose.
The participant has completed at least 2 cycles of study drug and have at least 1 postbaseline tumor imaging assessment.

Participants with evidence of progressive disease (PD), as defined by RECIST v1.1, continue to receive study drug if the participant is deriving benefit from continuing study drug. The following criteria must be met in order to provide assurance that the participant is not exposed to an unreasonable risk:
Absence of symptoms and signs indicating clinically significant progression of disease.
No decline in ECOG PS or Karnofsky Performance Status (KPS).
Absence of symptomatic rapid disease progression requiring urgent medical intervention (eg, symptomatic pleural effusion, spinal cord compression, etc).

At the time of radiographic progression, participants are reconsented and provided with details of all approved therapies, and potential clinical benefit, that the participant may be foregoing in order to continue receiving Formula (I).

Doses are increased no more than 50% of the previous dose beyond DL3 and intermediate doses may be explored. The MTD is the highest DL with no more than 35% of participants who have an AE that meets the criteria for a DLT. A participant may be replaced if they do not receive at least 75% of the planned dose during the DLT evaluation period for reasons other than a TRAE or if they discontinue the study for non-DLT reasons during the DLT evaluation period.

### Dose Escalation Decisions—Phase 1, Part A and B

In general, dose escalation or expansion proceeds according to the i3+3.

If any of the participant/study stopping criteria are met within a dose cohort, further enrollment into that cohort does not proceed until all safety data from participants at that dose are reviewed. If the decision is made not to initiate a new cohort before all planned prior cohorts are completed, it is not be regarded as premature study termination. Following the review of safety data, one of the following recommendations is made:
To continue with the study as planned (ie, there are no significant safety concerns) or with appropriate study amendments to address emerging safety risks.
To continue with the study but at a dose between the current dose and the next planned dose; or at a dose between the current dose and the previous lower dose.
To continue with the study but expand either the current cohort, or a higher/lower dose cohort, by an appropriate number of participants for more detailed safety evaluation.
To terminate the study.

### Definition of Dose-limiting Toxicities

For an AE to qualify as a DLT it must be causally related to Formula (I) (not all related AEs are regarded as a DLT). A DLT is defined as any of the following occurring during Cycle 1 of a DL and regarded to be related to Formula (I). The Common Terminology Criteria for Adverse Events (CTCAE) v5.0 is used to assess toxicities/AEs.

### Hematological Dose-Limiting Toxicities

Absolute neutrophil count (ANC)<500/mm³ for >7 days
Febrile neutropenia (a disorder characterized by an ANC<1,000/mm³ and a single temperature 38.3° C. or a sustained temperature of 38.0° C. for >1 hour); or Grade 3 neutropenia that lasts for >7 days with or without drug intervention
Platelets <25,000/mm³
Grade 3 hemorrhage associated with thrombocytopenia of ≥Grade 3

### Nonhematologic Dose-Limiting Toxicities

Nonhematologic Grade 3 or Grade 4 toxicity, including nausea and vomiting refractory to antiemetics. The following are not considered DLTs:
   Grade 3 nausea or vomiting of <48 hours duration.
   Grade 3 fatigue for <1 week.
   Grade 3 or higher electrolyte abnormality (excluding hyperphosphatemia) that lasts for ≤72 hours, is not clinically complicated, and resolves spontaneously or responds to conventional medical interventions.
   Grade 3 or higher amylase or lipase elevation that is not associated with symptoms or clinical manifestations of pancreatitis.
Grade 3 hyperphosphatemia (severe or medically significant but not immediately life-threatening; hospitalization or prolongation of existing hospitalization indicated) regardless of resolution with phosphate binders will be considered a DLT.
Hy's law:
   Aspartate aminotransferase (AST) or alanine aminotransferase (ALT)>3×upper limit of normal (ULN) and
   A total bilirubin >2×ULN and
   Alkaline phosphatase <2×ULN and
   No other reason for liver injury.
Any AST or ALT elevation >8×ULN regardless of duration and AST or ALT evaluation 5 to 8×ULN that persists for >2 weeks—all regardless of the presence or absence of liver metastasis
Dose delay of >14 days for the study drug due to an AE.
Any death not clearly due to the underlying disease or extraneous causes.

### Miscellaneous Dose-Limiting Toxicities

In case an unexpected drug-related toxicity is seen more frequently, this toxicity may be declared a DLT for the remainder of the study.

Any toxicity thought to be related to Formula (I) that warrants withholding the drug for >7 days during the DLT evaluation period. Such toxicities might be Grade 1 or Grade 2 toxicities which interfere with the activities of daily life (eg, long-lasting fatigue or anorexia), making a dose interruption/reduction necessary in order to ensure the participant's compliance. Examples may include Grade 2 nail loss or Grade 3 paronychia that does not improve after 4 weeks of supportive treatment.

For certain toxicities such as laboratory assessments without a clear clinical correlation (e.g., lipase increase without signs of a clinical pancreatitis; or Grade 2 hyperphosphatemia that does not recover to ≤Grade 1 within 7 days after treating with phosphate binders and diet modification), it is determine whether that AE should be assessed as a DLT necessitating dose reduction.

The first dosing cohorts (DL1 and DL2) utilizes a stricter definition for DLT than DL3 and above: any ≥Grade 2 TRAE constitutes a DLT for DL1 and DL2. If DL1 or DL2 are expanded due to this stricter DLT definition, further dose escalation decisions are made using the same DLT definitions used for DL3 and above (ie, no longer use ≥Grade 2 TRAEs as the definition for DLTs).

Results from the dose escalation and dose expansion cohorts is analyzed for safety, as well as potential relationships between dose, PK, biomarkers of target engagement, and preliminary antitumor activity of Formula (I). The RP2D is selected using the totality of the Phase 1 data.

### Phase 2: Cohort Expansion

Once an MTD is determined and an RP2D selected, participants are enrolled in Phase 2 expansion cohorts to further characterize preliminary efficacy, safety, and tolerability in the following cohorts:
1. Participants with locally advanced/metastatic urothelial carcinoma previously treated with an FGFR inhibitor who now have defined FGFR3 resistance mutations to any FGFR inhibitors:
   Participants must have had prior approved therapies including platinum-based chemotherapy, an immune checkpoint inhibitor, and an antibody-drug conjugate, unless the participant will not tolerate any of these approved agents, or the participant refuses any/all these therapies.
   Participants with urothelial carcinoma will be screened and evaluated for eligible resistance mutations using the CTA that assesses somatic mutations in ctDNA from plasma. Participants with an eligible gene alteration based on an authorized/approved or Clinical Laboratory Improvement Amendments (CLIA) (or regional equivalent) validated local test may be enrolled and a specimen must be submitted for central testing with the CTA. Confirmation is not necessary for these participants before starting Formula (I).
      Participants who discontinued a previous FGFR inhibitor for toxicity may be eligible if they have evidence of an activating FGFR3 alteration and a known FGFR3 acquired resistance mutation in the CTA or an authorized/approved or CLIA (or regional equivalent) validated local test.
2. Participants with locally advanced/metastatic urothelial carcinoma with activating FGFR3 gene alterations who have not previously received an FGFR inhibitor.
   Participants must have had prior approved therapies including platinum-based chemotherapy, an immune checkpoint inhibitor, and an antibody-drug conjugate, unless the participant will not tolerate any of these approved agents, or the participant refuses any/all these therapies.
   Participants with urothelial carcinoma will be screened and evaluated for eligible activating mutations and rearrangements with the CTA. Participants with an eligible gene alteration based on an authorized/approved or CLIA (or regional equivalent) validated local test may be enrolled and a specimen must be submitted for central testing with the CTA. Confirmation is not necessary for these participants before starting Formula (I).
3. Participants with any locally advanced/metastatic solid tumor with an activating FGFR3 gene alteration who have not previously received an FGFR inhibitor.
   Participants must have received prior approved therapies unless, the participant will not tolerate the approved agents, or the participant refuses any/all these therapies.
   Participants will be enrolled based on the results of an FDA authorized/approved companion diagnostic (CDx) or a CLIA (or regional equivalent) validated local test performed in a certified laboratory. A blood specimen must be submitted to the central laboratory for analysis using the CTA. Confirmation is not necessary for these participants before starting Formula (I).
   With medical monitor approval, participants who progressed on a prior FGFR inhibitor may be enrolled if they have an activating FGFR3 alteration and a known FGFR3 acquired resistance mutation in the CTA or an authorized/approved or CLIA (or regional equivalent) validated local test.

For Phase 2, approximately 110 participants are enrolled in each of Cohorts 1 and 2. A Simon's 2-Stage design is used for enrollment. In the first stage, 39 participants are accrued and if there are 6 or fewer responses, enrollment to the cohort is stopped for futility. Otherwise, 71 additional participants are accrued for a total of 110 participants. Responses are defined as CR or PR by RECIST v1.1 at any time while on therapy; for Cohort 1, clinical benefit (prolonged SD of ≥6 months while on therapy) counts as a response.

Cohort 3 is exploratory, and approximately 40 participants are enrolled in a single stage.

### Study Stopping Criteria

The study may be terminated at any time if the incidence and severity of AEs suggest that the risk-benefit to participants is no longer appropriate to continue the study. Examples include, but are not limited to, cases of severe liver toxicity without an underlying cause or unexplained participant deaths not due to disease progression or extraneous causes.

### Study Duration

All enrolled participants receive Formula (I) daily until disease progression, death, unacceptable toxicity, withdrawal of participant's consent for treatment, withdrawal from the study, or study termination, whichever comes first.
Planned For Phase 1, the total number of participants enrolled depends on the Sample Size number of escalation cohorts and the number of dose expansion and cohorts evaluated. Up to 50 participants could be enrolled in Parts A Treatment and B combined (up to 30 participants in Part A and up to Group(s): 20 participants in Part B).
   For Phase 2, approximately 110 participants are enrolled in each of Cohorts 1 and 2. A Simon's 2-stage design is used for enrollment. In the first stage, 39 participants are accrued and if there are 6 or fewer responses, enrollment to the cohort is stopped for futility. Otherwise, 71 additional participants are accrued for a total of 110 participants. The null hypothesis is rejected if 23 or more responses are observed in 110 participants. This design yields a 1-sided type I error rate of 5% and a power of 80% when the true response rate is 25%. Responses are defined as complete response (CR) or partial response (PR) by RECIST v1.1 at any time while on therapy; for Cohort 1, clinical benefit (prolonged stable disease [SD] of ≥6 months while on therapy) counts as a response.
      Cohort 3 is exploratory, and approximately 40 participants are enrolled in a single stage.
Target Phase 1, Part A: Participants with any advanced solid tumor who have Population: exhausted all standard treatment options.
   Phase 1, Part B: Participants with locally advanced/metastatic solid tumor and an FGFR3 activating gene alteration for whom an FGFR3 targeted therapy would be appropriate. Expansion at the MTD will enroll up to 10 participants with locally advanced/metastatic urothelial carcinoma with an FGFR3 activating gene alteration who have not previously received an FGFR inhibitor.
   Phase 2, Cohort 1: Participants with locally advanced/metastatic urothelial carcinoma previously treated with an FGFR inhibitor who now have defined FGFR3 resistance mutations.
   Phase 2, Cohort 2: Participants with locally advanced/metastatic urothelial carcinoma with defined activating FGFR3 gene alterations who have not previously received an FGFR inhibitor.
   Phase 2, Cohort 3: Participants with any locally advanced/metastatic solid tumor with defined activating FGFR3 gene alterations who have not previously received an FGFR inhibitor.
Eligibility Inclusion Criteria for Phase 1, Part A Criteria:
   1. Male and female participants who are 18 years of age or older on the day of signing the Informed Consent Form (ICF).
   2. Life expectancy >12 weeks.
   3. Ability to understand and sign the ICF and comply with study procedures.
   4. ECOGPS≤1.
   5. Participants with any histologically confirmed advanced solid tumor who have exhausted standard therapeutic options.
   6. Ability to swallow capsules.
   7. Disease evaluable by RECIST v1.1.
   8. Adequate organ and bone marrow function as demonstrated by the following:
      a. Absolute neutrophil count (ANC)≥1500/mm³.
      b. Platelet count ≥75,000/mm³.
      c. International normalized ratio (INR)≤1.5×upper limit of normal (ULN).
         i. Participants treated with anticoagulants (eg, warfarin or heparin) will be allowed to participate provided no prior evidence of an underlying abnormality in these parameters exists. Close monitoring of at least weekly evaluations will be performed until INR is stable based on a predose measurement as defined by the local standard of care.
      d. Total bilirubin ≤1.5×ULN. Documented Gilbert syndrome is allowed if total bilirubin is mildly elevated (<6 mg/dL).
      e. Alanine aminotransferase (ALT) and aspartate aminotransferase (AST)≤2.5×ULN (≤5×ULN for participants with liver involvement of their cancer).
      f. Serum albumin >2 g/dL.
      g. Glomerular filtration rate (GFR)≥60 mL/min/1.73 m² either directly measured via 24-hour urine collection or calculated using the Cockroft-Gault formula.
   9. Participants and their partners should practice contraception and reproduction restrictions of the study, as follows:
      a. Female participants of non-childbearing potential (ie, surgically sterile with a hysterectomy and/or bilateral oophorectomy; or chemically sterile; or ≥12 months of amenorrhea in the absence of chemotherapy, anti-estrogens, or ovarian suppression) do not need to undergo pregnancy testing.
   10. Female participants of child-bearing potential must have a documented negative pregnancy test within 7 days prior to starting Formula (I).
      a. Female participants of child-bearing potential and all male participants must agree to use highly effective contraception prior to study entry and up to 120 days after the last dose of Formula (I).
   11. Negative test result for coronavirus disease 2019 (COVID-19) by either a polymerase chain reaction (PCR)-based test within 48 hours or a rapid-antigen test within 24 hours prior to starting Formula (I); and fulfills COVID-19 vaccination requirements as per local site regulations (if any). Participants with a positive test result for COVID-19 infection at Screening who fulfil all other study eligibility criteria can be rescreened following test normalization and clinical recovery.
   Inclusion Criteria for Phase 1, Part B
   1. Male and female participants who are 18 years of age or older on the day of signing the ICF.
   2. Life expectancy >12 weeks.
   3. Ability to understand and sign the ICF and comply with study procedures.
   4. ECOGPS≤1.
   5. Participants with any histologically confirmed advanced solid tumor who have exhausted standard therapeutic options.
      a. The tumor must have an eligible FGFR3 gene mutation or fusion diagnosed by any authorized/approved or CLIA (or regional equivalent) validated local test performed in a certified laboratory facility.
      b. A specimen must be submitted for analysis using the CTA, but the results of the CTA do not need to be available before starting Formula (I).
      c. Any number of prior therapies, including prior FGFR inhibitors, are permitted.
      d. At the MTD, up to 10 participants with metastatic urothelial carcinoma with eligible FGFR3 gene alterations identified by the CTA or any authorized/approved CLIA (or regional equivalent) validated local test who have not received a prior FGFR inhibitor.
   6. Ability to swallow capsules.
   7. At least 1 measurable lesion by RECIST v1.1.
   8. Adequate organ and bone marrow function as demonstrated by the following:
      a. ANC≥1500/mm³.
      b. Platelet count ≥75,000/mm³.
      c. INR≤1.5×ULN.
         i. Participants treated with anticoagulants (eg, warfarin or heparin) will be allowed to participate provided no prior evidence of an underlying abnormality in these parameters exists. Close monitoring of at least weekly evaluations will be performed until INR is stable based on a predose measurement as defined by the local standard of care.
      d. Total bilirubin ≤1.5×ULN. Documented Gilbert syndrome is allowed if total bilirubin is mildly elevated (<6 mg/dL).
      e. ALT and AST≤2.5×ULN (≤5×ULN for participants with liver involvement of their cancer).
      f. Serum albumin >2 g/dL.
      g. GFR≥45 mL/min/1.73 m² either directly measured via 24-hour urine collection or calculated using Cockroft-Gault formula.
   9. Participants and their partners should practice contraception and reproduction restrictions of the study, as follows:
      a. Female participants of non-childbearing potential (ie, surgically sterile with a hysterectomy and/or bilateral oophorectomy; or chemically sterile; or ≥12 months of amenorrhea in the absence of chemotherapy, anti-estrogens, or ovarian suppression) do not need to undergo pregnancy testing.
         Female participants of child-bearing potential must have a documented negative pregnancy test within 7 days prior to starting Formula (I).
      b. Female participants of child-bearing potential and all male participants must agree to use highly effective contraception prior to study entry and up to 120 days after the last dose of Formula (I).
   10. Negative test result for COVID-19 by either a PCR-based test within 48 hours or a rapid-antigen test within 24 hours prior to starting Formula (I); and fulfils COVID-19 vaccination requirements as per local site regulations (if any). Participants with a positive test result for COVID-19 infection at Screening who fulfil all other study eligibility criteria can be rescreened following test normalization and clinical recovery.
   Inclusion Criteria for Phase 2
   1. Male and female participants who are 12 years of age or older on the day of signing the ICF.
   2. Life expectancy >12 weeks.
   3. Ability to understand and willingness to sign the ICF. For participants under 18 years of age (or country equivalent), a parent/legal guardian with the ability to understand and sign the informed consent and the child with the ability to understand and sign the Assent Form.
   4. ECOG PS 0 to 2. KPS>70 for participants aged 12 to 17 years.
   5. Participants must have a histologically confirmed locally advanced/metastatic tumor in 1 of the following categories:
      a. Urothelial carcinoma with an eligible FGFR3 gene mutation or rearrangement who have progressed on a prior FGFR inhibitor and presence of a resistance mutation or other kinase domain mutation likely to respond to Formula (I) identified using the CTA or an FDA authorized/approved CDx or a CLIA (or regional equivalent) validated local test performed in a certified laboratory.
      b. Urothelial carcinoma with an eligible FGFR3 gene mutation or rearrangement identified by the central CTA or an FDA authorized/approved CDx or a CLIA (or regional equivalent) validated local test performed in a certified laboratory who has not received a prior FGFR inhibitor.
      c. Any solid tumor with an eligible FGFR3 gene mutation or rearrangement identified via an authorized/approved or CLIA (or regional equivalent) validated local test result in a certified laboratory.
         i. With medical monitor approval, participants who progressed on a prior FGFR inhibitor may be enrolled if they have a documented FGFR3 resistance mutation or other kinase domain mutation for which Formula (I) is likely to be active based on preclinical studies.
   6. Ability to swallow capsules.
   7. At least 1 measurable lesion by RECIST v1.1.
   8. Adequate organ and bone marrow function as demonstrated by the following:
      a. ANC≥1500/mm³.
      b. Platelet count ≥75,000/mm³.
      c. INR≤1.5×ULN.
         i. Participants treated with anticoagulants (eg, warfarin or heparin) will be allowed to participate provided no prior evidence of an underlying abnormality in these parameters exists. Close monitoring of at least weekly evaluations will be performed until INR is stable based on a predose measurement as defined by the local standard of care.
      d. Total bilirubin ≤1.5×ULN. Documented Gilbert syndrome is allowed if total bilirubin is mildly elevated (<6 mg/dL).
      e. ALT and AST≤2.5×ULN (≤5×ULN for participants with liver involvement of their cancer).
      f. Serum albumin >2 g/dL.
      g. GFR≥45 mL/min/1.73 m² either directly measured via 24-hour urine collection or calculated using the Cockroft-Gault formula.
   9. Participants and their partners should practice contraception and reproduction restrictions of the study, as follows:
      a. Female participants of non-childbearing potential (ie, surgically sterile with a hysterectomy and/or bilateral oophorectomy; or chemically sterile; or ≥12 months of amenorrhea in the absence of chemotherapy, anti-estrogens, or ovarian suppression) do not need to undergo pregnancy testing.
   10. Female participants of child-bearing potential must have a documented negative pregnancy test within 7 days prior to starting Formula (I).
      a. Female participants of child-bearing potential and all male participants must agree to use highly effective contraception prior to study entry and up to 120 days after the last dose of Formula (I).
   11. Negative test result for COVID-19 by either a PCR-based test within 48 hours or a rapid-antigen test within 24 hours prior to starting Formula (I); and fulfils COVID-19 vaccination requirements as per local site regulations (if any). Participants with a positive test result for COVID-19 infection at Screening who fulfil all other study eligibility criteria can be rescreened following test normalization and clinical recovery.
   Study Exclusion Criteria
   1. Participant received chemotherapy, targeted therapy, immunotherapy, or an investigational therapy within 2 weeks or 5 half-lives (within 6 weeks for nitrosoureas and mitomycin) before the first dose of study drug.
   2. Participant has not recovered from reversible toxicity of prior anticancer therapy (except toxicities that are not clinically significant including, but not limited to, alopecia, skin discoloration, or Grade 1 neuropathy).
   3. Had major surgery within 4 weeks prior to enrollment.
   4. Any reason thatr, would substantially impair the ability of the participant to comply with study procedures and increase the risk to the participant. Examples include poorly controlled diabetes (glycated hemoglobin [HbA1c]>8%) and ongoing active infection requiring intravenous (IV) antibiotics.
   5. Females who are pregnant, breastfeeding, or planning to become pregnant within 120 days after the last dose of Formula (I) and males who plan to father a child while enrolled in this study or within 120 days after the last dose of Formula (I).
   6. Has impaired wound healing capacity defined as skin/decubitus ulcers, chronic leg ulcers, known gastric ulcers, or unhealed incisions.
   7. Has a serum phosphorus level >ULN during screening (within 14 days of treatment and prior to Cycle 1, Day 1) that remains >ULN despite medical management with phosphate binders.
   8. Any ocular condition likely to increase the risk of eye toxicity, including:
      a. History of or current evidence of central serous retinopathy (CSR; including ≥Grade 2 CSR while receiving a prior FGFR inhibitor) or retinal vascular occlusion (RVO).
      b. Active wet, age-related macular degeneration (AMD).
      c. Diabetic retinopathy with macular edema.
      d. Uncontrolled glaucoma (per local standard of care).
   9. History of or current uncontrolled cardiovascular disease including:
      a. Unstable angina, myocardial infarction, or known congestive heart failure Class II to IV within the preceding 12 months.
      b. Cerebrovascular accident or transient ischemic attack within the preceding 3 months.
      c. Pulmonary embolism within the preceding 2 months.
   10. Active, symptomatic, or untreated brain metastases.
      a. Prior brain metastases treated at least 3 weeks prior to signing the full-study ICF or that are clinically and radiographically stable for at least 1 month prior to Cycle 1, Day 1 and do not require chronic corticosteroid treatment are allowed.
   11. Gastrointestinal disorders that will affect oral administration or absorption of Formula (I).
   12. Known history of human immunodeficiency virus (HIV) infection, or active hepatitis B or C infection.
      a. Participants with a history of HIV on antiviral therapy with undetectable viral load by PCR are allowed.
      b. Participants with a history of hepatitis B virus infection with positive hepatitis B surface antibody, or positive hepatitis B core antibody with a negative PCR test, are allowed.
      c. Participants with hepatitis C infection previously treated with antiviral therapy and test negative for hepatitis C virus by PCR are allowed.
   13. History of a second primary malignancy within 3 years of signing the ICF (except definitively treated early-stage cancer such as resected skin cancers and/or completely resected prostate cancer).
   14. Known allergy to Formula (I) or any excipients of the formulated product.
   15. Participants taking strong inhibitors and/or inducers of cytochrome P450 (CYP) 3A4 enzymes are prohibited. Strong inhibitors and strong inducers of CYP3A are prohibited from 2 weeks prior to entry into the study until 1 week after administration of the last dose of Formula (I). Strong inhibitors of cytochrome P450 (CYP) 3A4: Boceprevir, clarithromycin, cobicistat, danoprevir and ritonavir, elvitegravir and ritonavir, grapefruit juice, idelalisib, indinavir and ritonavir, itraconazole, ketoconazole, lopinavir and ritonavir, nefazodone, nelfinavir, paritaprevir and ritonavir and (ombitasvir and/or dasabuvir), posaconazole, ritonavir, saquinavir and ritonavir, telaprevir, tipranavir and ritonavir, telithromycin, troleandomycin, voriconazole. Strong inducers of cytochrome P450 (CYP) 3A4: Apalutamide, carbamazepine, enzalutamide, mitotane, phenytoin, rifampin, St. John's wort.
      a. Strong inhibitors are drugs that increase the AUC of sensitive index substrates ≥5-fold.
      b. Strong inducers are drugs that decrease the AUC of sensitive index substrates by ≥80%.
      Abbreviation: AUC=area under the concentracion time curve, CYP=cytochrome P450. Source: US Food and Drug Administration. Drug Development and Drug Interactions. Table of Substrates, Inhibitors and Inducers. US Food and Drug Administration. Drug Development and Drug Interactions. Table of Substrates, Inhibitors and Inducers. 10 Mar. 2020. www.fda.gov/drugs/drug-interactions-labeling/drug-development-and-drug-interactions-table-substrates-inhibitors-and-inducers.
      c. Participants who can switch to a similar medication without a CYP3A4 interaction will require a washout period of 2 weeks prior to starting Formula (I).
Study Formula (I) capsules in the following strengths: 5 mg and 10 mg Drug(s): (based on the free base). The capsules are packaged in high-density polyethylene bottles for the study, each containing 30 capsules per bottle. Formula (I) capsules contain the study drug as the active ingredient (as free base or as a pharmaceutically acceptable salt) and microcrystalline cellulose as an inactive ingredient.
   Participants are instructed to take Formula (I) at the same time of day each day, with food or within 30 to 60 minutes after food or a small meal.

### Schedule of Assessments for Phase 1 Part a and Phase 1, Part B:

| | | | | | | | | | Safety | |
|---|---|---|---|---|---|---|---|---|---|---|
| | | | | | | | | | Follow-up^{b} | |
| | | | | | | | | | 28 Days | Survival |
| Screening | | | | | | Cycles 3 | Cycle 13 | EOT^{a} | After | Follow-up^{c} |
| Day −28 | Cycle 1^{a} | | | Cycle 2^{a} | | to 12^{a} | Onwards^{a} | Final | Last | Every |
| to −1 | C1D1 | C1D8 | C1D15 | C2D1 | C2D15 | CXD1 | CXD1 | Visit | Dose | 3 Months |
| Time window | | | | | | | | | | |
| — | — | ±1 day | ±2 days | ±2 days | ±2 days | ±3 days | ±3 days | ±7 days | +7 days | ±15 days |

Administrative

| | | | | | | | | | | |
|---|---|---|---|---|---|---|---|---|---|---|
| Informed | X | | | | | | | | | |
| consent | | | | | | | | | | |
| Contact IRT | X | X | | X | | X | X | X | | |
| Inclusion/ | X | | | | | | | | | |
| exclusion | | | | | | | | | | |
| criteria | | | | | | | | | | |
| Demographics | X | | | | | | | | | |
| Medical | X | | | | | | | | | |
| history and | | | | | | | | | | |
| oncology | | | | | | | | | | |
| history | | | | | | | | | | |
| Prior and | X | X^{d} | X | X | X | X | X | X | | |
| concomitant | | | | | | | | | | |
| medication | | | | | | | | | | |
| review | | | | | | | | | | |
| Dispense | | X | | X | | X | X | | | |
| Formula (I) | | | | | | | | | | |
| and | | | | | | | | | | |
| distribute | | | | | | | | | | |
| reminder | | | | | | | | | | |
| cards^{e} | | | | | | | | | | |
| Assess | | | | X | | X | X | | | |
| compliance | | | | | | | | | | |
| Safety | | | | | | | | | X | |
| follow-up | | | | | | | | | | |
| Survival | | | | | | | | | | X |
| status | | | | | | | | | | |
| Subsequent | | | | | | | | | X | X |
| anticancer | | | | | | | | | | |
| therapy | | | | | | | | | | |

Safety assessments

| | | | | | | | | | | |
|---|---|---|---|---|---|---|---|---|---|---|
| COVID-19 | X | X^{d} | X | X | X | X | X | X | X | |
| signs, | | | | | | | | | | |
| symptoms, | | | | | | | | | | |
| and recent | | | | | | | | | | |
| exposure; | | | | | | | | | | |
| and testing^{f} | | | | | | | | | | |
| Height | X | | | | | | | | | |
| Weight | X | X^{d} | | X | X | X | X | X | X | |
| Physical | X | X^{d} | | X | X | X | X | X | X | |
| examination | | | | | | | | | | |
| and vital | | | | | | | | | | |
| signs^{g} | | | | | | | | | | |
| ECOG PS | X | X | | | X | | X | X | X | |
| ECG (and as | X | Xⁱ | | Xⁱ | | | | | | |
| indicated)^{h} | | | | | | | | | | |
| Ophthalmologic | X | | | | | | X^{k} | X^{k} | | |
| examination | | | | | | | | | | |
| (and as | | | | | | | | | | |
| indicated)^{j} | | | | | | | | | | |
| Amsler | | X^{d} | | | X | | X^{l} | X^{l} | | |
| grid | | | | | | | | | | |
| screening | | | | | | | | | | |
| AE review | X | X | X | X | X | X | X | X | X | X |
| and | | | | | | | | | | |
| evaluation | | | | | | | | | | |

Local laboratory tests

| | | | | | | | | | |
|---|---|---|---|---|---|---|---|---|---|
| Hematology^{m} | Xⁿ | X^{d} | | X | X | | X | X | X |
| Serum | Xⁿ | X^{d} | X | X | X | X | X | X | X |
| chemistry^{o} | | | | | | | | | |
| Pregnancy | Xⁿ | X^{d} | | | X | | X | X | X |
| testing | | | | | | | | | |
| (female | | | | | | | | | |
| participants of | | | | | | | | | |
| childbearing | | | | | | | | | |
| potential only)^{p} | | | | | | | | | |
| Viral | Xⁿ | | | | | | | | |
| hepatitis | | | | | | | | | |
| serology^{q} | | | | | | | | | |

Efficacy assessments

| | | | | |
|---|---|---|---|---|
| Radiologic/ | X^{s} | X^{t} | X^{u} | X^{v} |
| imaging | | | | |
| assessment^{r} | | | | |

Sample collection (for central processing)

| | | | | | | | | |
|---|---|---|---|---|---|---|---|---|
| PK sampling^{a} | | X^{w} | X^{w} | X^{w} | X^{w} | X^{w} | | |
| | | | | | | (C3D1 | | |
| | | | | | | only) | | |
| Pharmacodynamic | | X^{d} | X | X | | | | |
| sampling | | | | | | | | |
| (blood and | | | | | | | | |
| urine)^{x} | | | | | | | | |
| ctDNA | X | | X | X | | X^{t} | X^{u} | X |
| sampling | | | | | | | | |
| (blood and | | | | | | | | |
| urine)^{y} | | | | | | | | |
| Biomarker | X | | | X | | | | X^{z} |
| sampling | | | | | | | | (optional) |
| (tissue/skin)^{z} | | | | | | | | |
| Archived | X | | | | | | | |
| tumor | | | | | | | | |
| tissue^{aa} | | | | | | | | |

1 cycle = 4 weeks (28 days).
^{a}Unscheduled visits may be held at any time, and appropriate clinical assessments and laboratory measurements will be performed based on AEs or other findings. Data and results should be entered in the electronic case report form. A PK blood sample is to be collected at any unscheduled visit due to an AE. The last study drug administration date and time, and the PK sampling time (24-hour clock time) should be accurately recorded.
^{b}Participants will be followed up for safety (via telephone or video contact) 28 days (+7 days) after the last dose of study drug. Subsequent anticancer therapy will also be collected.
^{c}Partcipants will be followed up for survival every 3 months (±15 days) until the participant withdraws consent for further participation, is lost to follow-up, has died, or study closure. Contact may be performed via telephone, video, email, or certified mail. Subsequent anticancer therapy will also be collected.
^{d}The following assessments and local laboratory samples required at C1D1 may be performed within 2 days prior to C1D1: Concomitant medicine review; COVID-19 signs, symptoms, and recent exposure; physical examination including weight; Amsler grid screening; laboratory assessments (hematology, serum chemistry, and pregnancy testing); and pharmacodynamic sampling (blood and urine).
^{e}Distribution of reminder cards should be documented in the source.
^{f}COVID-19 signs, symptoms, and recent exposure will be assessed at each time point indicated. A negative COVID-19 test result is required by either a PCR- based test within 48 hours or a rapid-antigen test within 24 hours prior to starting Formula (I) and will be performed thereafter as necessary, per local guidance or practice.
^{g}A full physical examination will be performed at Screening only. Targeted physical examinations will be performed from C1D1 onwards; however, there is no planned physical examination on C1D8. Vital signs include body temperature, respiratory rate, sitting radial pulse rate, and sitting systolic and diastolic blood pressures.
^{h}ECGs will be transmitted from the site electronically to the sponsor-designated central vendor for “collect and hold”.
ⁱTriplicate 12-lead ECGs will be performed with PK blood draws on C1D1 (predose, and 30 minutes and 1, 2, 4, 8, and 24 hours postdose) and C1D15 (predose, and 30 minutes and 1, 2, 4, 8, and 24 hours postdose). When an ECG measurement is scheduled at the time of a PK blood sample collection, the ECG measurements should be completed immediately prior to collection of the PK blood sample.
^{j}Evaluation includes noncontact intraocular pressure, slit lamps, fundus examinations, and optical coherence tomography. Changes in vision during treatment should be referred for ophthalmologic examination.
^{k}Ophthalmologic examination will be performed at Screening, C3D1 (±7 days), on Day 1 of every 3 cycles thereafter (ie, C6D1, C9D1, C12D1, etc; ±7 days), and as clinically indicated.
^{l}Amsler grid screening will be conducted on all CxD1 visits where ophthalmology examinations are not performed (ie, C1D1, C2D1, C4D1, C5D1, C7D1, C8D1, etc).
^{m}Complete blood count with platelets and INR (INR only at Screening and for monitoring, as needed, for anticoagulation).
ⁿIf Screening laboratory assessments are performed within 7 days of C1D1 and are found to be within the normal local laboratory limits, these results may be used in place of the local laboratory assessments scheduled on C1D1.
^{o}Albumin, ALT, alkaline phosphatase, AST, bicarbonate, bilirubin (direct, indirect, and total), blood urea nitrogen/urea nitrogen, calcium, chloride, creatinine, gamma-glutamyl transpeptidase, glucose, lactate dehydrogenase, phosphorus, potassium, total protein, and sodium.
^{p}Urine pregnancy testing, for female participants of childbearing potential only, will be performed at Screening and on Day 1 of each cycle, as per local requirements. A positive urine pregnancy test result at Screening will be confirmed by serum testing.
^{q}Hepatitis B virus and Hepatitis C virus testing required at Screening.
^{r}The preferred tumor imaging assessment modality is CT with IV contrast; however, non-contrast CT of the chest and MRI of the abdomen and pelvis are permitted if IV contrast is clinically contraindicated. Screening and postbaseline efficacy assessments will be performed using the same imaging method.
^{s}Baseline tumor assessment will be completed within 28 days prior to the first dose of study drug. It is recommended to perform CT of the chest, abdomen, pelvic cavity, and any other location where disease is present at baseline; additional tumor imaging evaluation must be performed if clinically indicated or if suspicious lesions are identified in other areas. Baseline brain MRI is not required unless there is a prior history of brain metastases or neurologic symptoms suggestive of new brain metastases.
^{t}Every 8 weeks (C3D1, C5D1, C7D1, C9D1, C11D1, and C13D1) with a window of ±7 days.
^{u}Every 12 weeks, starting at C13D1 (C16D1, C19D1, etc) with a window of ±7 days.
^{v}If study drug is discontinued due to reasons other than documented disease progression (eg, toxicity), radiologic/imaging assessments should continue as planned until initiation of subsequent treatment or withdrawal from the study.
^{w}PK blood sample collection on C1D1 (predose, and 30 minutes and 1, 2, 4, 8, and 24 hours postdose), C1D15 (predose, and 30 minutes and 1, 2, 4, 8, and 24 hours postdose), C2D1 (predose), C2D15 (predose), and C3D1 (predose). Formula (I) should be given with food or within 30 to 60 minutes after food or a small meal (eg, eggs, toast, yogurt, or small sandwich) and the date and time of the meal are to be recorded in the eCRF. For all PK samples, the actual study drug administration time and PK sampling time (24-hour clock time) should be accurately recorded.
^{x}Blood and urine samples for circulating biomarkers will be collected predose at C1D1, C1D15, and C2D1. Blood biomarkers include, but are not limited to, FGF23, parathyroid hormone, calcitriol, FGF19, and tumor-derived exosomes. In pediatric participants (12 to 21 years of age), CXM may also be evaluated. Urine biomarkers include, but are not limited to, matrix metalloproteinase-1, matrix metalloproteinase-10, and FGF binding protein 1.
^{y}ctDNA (blood and urine) sampling is only required from participants with FGFR3 gene alterations in Phase 1, Part A and from all participants in Phase 1, Part B.
^{z}Biomarker sampling using 2 mm skin punch biopsies/tape strips will be collected at Screening and C2D1. Optional at EOT and as needed to assess skin toxicity.
^{aa}Archival tumor biopsy for participants in Phase 1, Part A with FGFR3 gene alterations only and all participants in Phase 1, Part B. An archival tumor biopsy sample must be provided, if available; however, no new biopsy is required if an archival sample is not available.
Abbreviations: AE = adverse event, ALT = alanine aminotransferase, AST = aspartate aminotransferase, COVID-19 = Coronavirus Disease 2019, CT computed tomography, ctDNA = circulating tumor DNA, CxDx = Cycle x, Day x, CXM = serum Collagen Type X, ECG = electrocardiogram, eCRF = electronic Case Report Form, ECOG PS = Eastern Cooperative Oncology Group Performance Status, EOT = End of Treatment, FGF = fibroblast growth factor, FGFR = fibroblast growth factor receptor, INR = international normalized ratio, IRT = interactive response technology, IV = intravenuous; MRI = magnetic resonance imaging, PCR = polymerase chain reaction, PK = pharmacokinetic.

### Schedule of Assessments for Phase 2:

| | | | | | | | | | Safety | |
|---|---|---|---|---|---|---|---|---|---|---|
| | | | | | | | | | Follow-up^{b} | |
| | | | | | | | | | 28 Days | Survival |
| Screening | | | | | | Cycles 3 | Cycle 13 | EOT^{a} | After | Follow-up^{c} |
| Day −28 | Cycle 1^{a} | | | Cycle 2^{a} | | to 12^{a} | Onwards^{a} | Final | Last | Every |
| to −1 | C1D1 | C1D8 | C1D15 | C2D1 | C2D15 | CXD1 | CXD1 | Visit | Dose | 3 Months |
| Time window | | | | | | | | | | |
| — | — | ±1 day | ±2 days | ±2 days | ±2 days | ±3 days | ±3 days | ±7 days | +7 days | ±15 days |

Administrative

| | | | | | | | | | | |
|---|---|---|---|---|---|---|---|---|---|---|
| Informed consent | X | | | | | | | | | |
| Contact IRT | X | X | | X | | X | X | X | | |
| Inclusion/exclusion | X | | | | | | | | | |
| criteria | | | | | | | | | | |
| Demographics | X | | | | | | | | | |
| Medical history and | X | | | | | | | | | |
| oncology history | | | | | | | | | | |
| Prior and | X | X^{d} | X | X | X | X | X | X | | |
| concomitant | | | | | | | | | | |
| medication review | | | | | | | | | | |
| Dispense Formula | | X | | X | | X | X | | | |
| (I) and distribute | | | | | | | | | | |
| reminder cards^{e} | | | | | | | | | | |
| Assess compliance | | | | X | | X | X | | | |
| Safety follow-up | | | | | | | | | X | |
| Survival status | | | | | | | | | | X |
| Subsequent | | | | | | | | | X | X |
| anticancer therapy | | | | | | | | | | |

Safety assessments

| | | | | | | | | | | |
|---|---|---|---|---|---|---|---|---|---|---|
| COVID-19 signs, | X | X^{d} | X | X | X | X | X | X | X | |
| symptoms, and | | | | | | | | | | |
| recent exposure; | | | | | | | | | | |
| and testing^{f} | | | | | | | | | | |
| Height (participants | X | X | | | | | X^{g} | X^{g} | | |
| age 12 to 21 years) | | | | | | | | | | |
| Height (participants | X | | | | | | | | | |
| age ≥22 years) | | | | | | | | | | |
| Weight | X | X^{d} | | X | X | X | X | X | X | |
| Physical | X | X^{d} | | X | X | X | X | X | X | |
| examination and | | | | | | | | | | |
| vital signs^{h} | | | | | | | | | | |
| ECOG PS | X | X | | | X | | X | X | X | |
| ECG (and as | X | | | | | | | | | |
| indicated)ⁱ | | | | | | | | | | |
| Ophthalmologic | X | | | | | | X^{k} | X^{k} | | |
| examination (and as | | | | | | | | | | |
| indicated)^{j} | | | | | | | | | | |
| Amsler grid | | X^{d} | | | X | | X^{l} | X^{l} | | |
| screening | | | | | | | | | | |
| AE review and | X | X | X | X | X | X | X | X | X | X |
| evaluation | | | | | | | | | | |

Local laboratory tests

| | | | | | | | | | |
|---|---|---|---|---|---|---|---|---|---|
| Hematology^{m} | Xⁿ | X^{d} | | X | X | | X | X | X |
| Serum chemistry^{o} | Xⁿ | X^{d} | X | X | X | X | X | X | X |
| Pregnancy testing | Xⁿ | X^{d} | | | X | | X | X | X |
| (female participants | | | | | | | | | |
| of childbearing | | | | | | | | | |
| potential only)^{p} | | | | | | | | | |
| Viral hepatitis | Xⁿ | | | | | | | | |
| serology^{q} | | | | | | | | | |

Efficacy assessments

| | | | | |
|---|---|---|---|---|
| Radiologic/imaging | X^{s} | X^{t} | X^{u} | X^{v} |
| assessment^{r} | | | | |

Sample collection (for central processing)

| | | | | | | | | |
|---|---|---|---|---|---|---|---|---|
| PK sampling^{a} | | X^{w} | | X^{w} | X^{w} | X^{w} | | |
| | | | | | | (C3D1 | | |
| | | | | | | only) | | |
| Pharmacodynamic | | X^{d} | X | X | | | | |
| sampling (blood and | | | | | | | | |
| urine)^{x} | | | | | | | | |
| ctDNA sampling | X | | X | X | | X^{t} | X^{u} | X |
| (blood and urine) | | | | | | | | |
| Biomarker sampling | X | | | | | | | X |
| (tissue/skin)^{y} | | | | | | | | |
| Archived tumor | X | | | | | | | |
| tissue^{z} | | | | | | | | |

1 cycle = 4 weeks (28 days).
^{a}Unscheduled visits may be held at any time and appropriate clinical assessments and laboratory measurements will be performed based on AEs or other findings. Data and results should be entered in the electronic case report form. A PK blood sample is to be collected at any unscheduled visit due to an AE. The last study drug administration date and time, and the PK sampling time (24-hour clock time) should be accurately recorded.
^{b}Participants will be followed up for safety (via telephone or video contact) 28 days (+7 days) after the last dose of study drug. Subsequent anticancer therapy will also be collected.
^{c}Participants will be followed up for survival every 3 months (±15 days) until the participant withdraws consent for further participation, is lost to follow-up, has died, or study closure. Contact may be performed via telephone, video, email, or certified mail. Subsequent anticancer therapy will also be collected.
^{d}The following assessments and local laboratory samples required at C1D1 may be performed within 2 days prior to C1D1: Concomitant medicine review; COVID-19 signs, symptoms, and recent exposure; physical examination including weight; Amsler grid screening; laboratory assessments (hematology, serum chemistry, and pregnancy testing); and pharmacodynamic sampling (blood and urine).
^{e}Distribution of reminder cards should be documented in the source.
^{f}COVID-19 signs, symptoms, and recent exposure will be assessed at each time point indicated. A negative COVID-19 test result is required by either a PCR- based test within 48 hours or a rapid-antigen test within 24 hours prior to starting Formula (I) and will be performed thereafter as necessary, per local guidance or practice.
^{g}Every 8 weeks, starting at C1D1 (C3D1, C5D1, C7D1, C9D1, etc).
^{h}A full physical examination will be performed at Screening only. Targeted physical examinations will be performed from C1D1 onwards; however, there is no planned physical examination on C1D8. Vital signs include body temperature, respiratory rate, sitting radial pulse rate, and sitting systolic and diastolic blood pressures.
ⁱTriplicate 12-lead ECG.
^{j}Changes in vision during treatment should be referred for ophthalmologic examination.
^{k}Ophthalmologic examination will be performed at Screening, C3D1 (±7 days), on Day 1 of every 3 cycles thereafter (ie, C6D1, C9D1, C12D1, etc; ±7 days), and as clinically indicated.
^{l}Amsler grid screening will be conducted on all CxD1 visits where ophthalmology examinations are not performed (ie, C1D1, C2D1, C4D1, C5D1, C7D1, C8D1, etc).
^{m}Complete blood count with platelets, INR (INR only at Screening and for monitoring, as needed, for anticoagulation).
ⁿIf Screening laboratory assessments are performed within 7 days of C1D1 and are found to be within the normal local laboratory limits, these results may be used in place of the local laboratory assessments scheduled on C1D1.
^{o}Albumin, ALT, alkaline phosphatase, AST, bicarbonate, bilirubin (direct, indirect, and total), blood urea nitrogen/urea nitrogen, calcium, chloride, creatinine, gamma-glutamyl transpeptidase, glucose, lactate dehydrogenase, phosphorus, potassium, total protein, and sodium.
^{p}Urine pregnancy testing, for female participants of childbearing potential only, will be performed at Screening and on Day 1 of each cycle, as per local requirements. A positive urine pregnancy test result at Screening will be confirmed by serum testing.
^{q}Hepatitis B virus and Hepatits C virus testing required at Screening.
^{r}The preferred tumor imaging assessment modality is CT with IV contrast; however, non-contrastCT of the chest and MRI of the abdomen and pelvis are permitted if IV contrast is clinically contraindicated. Screening and postbaseline efficacy assessments will be performed using the same imaging method. For participants enrolled in Phase 2, Cohorts 1 and 2 only, digital images of scans performed will be transmitted from the sites electronically to the sponsor-designated central radiology vendor for “collect and hold”.
^{s}Baseline tumor assessment will be completed within 28 days prior to the first dose of study drug. It is recommended to perform CT of the chest, abdomen, pelvic cavity, and any other location where disease is present at baseline; additional tumor imaging evaluation must be performed if clinically indicated or if suspicious lesions are identified in other areas. Baseline brain MRI is not required unless there is a prior history of brain metastases or neurologic symptoms suggestive of new brain metastases.
^{t}Every 8 weeks (C3D1, C5D1, C7D1, C9D1, C11D1, and C13D1) with a window of ±7 days.
^{u}Every 12 weeks, starting at C13D1 (C16D1, C19D1, etc) with a window of ±7 days.
^{v}If study drug is discontinued due to reasons other than documented disease progression (eg, toxicity), radiologic/imaging assessments should continue as planned until initiation of subsequent treatment or withdrawal from the study.
^{w}PK blood sample collection on C1D1 (1, 2, and 4 hours postdose), C2D1 (predose and 1, 2, and 4 hours postdose), C2D15 (predose), and C3D1 (predose). For all PK samples, the actual study drug administration time and PK sampling time (24-hour clock time) should be accurately recorded in the eCRF.
^{x}Blood and urine samples for circulating biomarkers will be collected predose at C1D1, C1D15, and C2D1. Blood biomarkers include, but are not limited to, FGF23, parathyroid hormone, calcitriol, FGF19, and tumor-derived exosomes. In pediatric participants (12 to 21 years of age), CXM may also be evaluated. Urine biomarkers include, but are not limited to, matrix metalloproteinase-1, matrix metalloproteinase-10, and FGF binding protein 1.
^{y}Biomarker sampling using 2 mm skin punch biopsies/tape strips will be collected at Screening, EOT, and as needed to assess skin toxicity.
^{z}An archival tumor biopsy sample must be provided, if available; however, no new biopsy is required if an archival sample is not available.
Abbreviations: AE = adverse event, ALT = alanine aminotransferase, AST = aspartate aminotransferase, CT = computed tomography, COVID-19 = Coronavirus Disease 2019, ctDNA = circulating tumor DNA, CxDx = Cycle x, Day x, ECG = electrocardiogram, ECOG PS = Eastern Cooperative Oncology Group Performance Status, EOT = End of Treatment, FGF = fibroblast growth factor, INR = international normalized ratio, IRT = Interactive Response Technology, IV = intravenuous, MRI = magnetic resonance imaging, PCR = polymerase chain reaction, PK = pharmacokinetic.

Screening is the interval between signing the ICF and the day the participant is enrolled in the study (Cycle 1, Day 1). The screening visit is to occur within 28 calendar days prior to the start of study drug administration.

For each participant, the treatment period continues in 4-week cycles (ie, 28 days) until disease progression, unless the participant fulfills 1 of the discontinuation of treatment criteria.

The safety follow-up period is the interval between the EOT visit and the scheduled follow-up visit, which should occur 28 to 35 days after the EOT visit (or after the last dose of study drug if the EOT visit was not performed).

Once a participant has completed the safety follow-up visit and/or starts a new anticancer therapy, the participant moves into the survival follow-up period and should be contacted by telephone, video, email, certified mail, or visit at least every 12 weeks to assess for survival status until death, withdrawal of consent, or the end of the study, whichever occurs first.
Primary Phase 1 (Parts A and B): Incidence of DLT events during the DLT Endpoint(s): evaluation period (Cycle 1 [28 days]).
   Phase 2: Investigator-assessed ORR, defined as either a CR or PR by RECIST v1.1.
Secondary Phase 1 (Parts A and B) and Phase 2:
Endpoint(s):
   Incidence of AEs characterized by study phase, cohort, seriousness, relationship to study drug, timing, and severity
   Changes in clinical laboratory parameters, vital signs, electrocardiogram (ECG) parameters, and physical examination status
   Single-dose and steady-state PK parameters, including but not limited to accumulation ratio, Cₘₐₓ, Tₘₐₓ, AUC₀₋ₗₐₛₜ, AUC_{Tau}, AUC_{0-∞} (after first dose only), Vd/F, CL/F, and t_{1/2}
   Additional secondary endpoint for Phase 1, Part B only:
      ORR
   Additional secondary endpoints for Phase 1, Part B and Phase 2:
      DOR
      DCR, defined as CR, PR, or SD for >12 weeks
      TTR
   Additional secondary endpoints for Phase 2 only:
      PFS in Cohorts 1 and 2
Exploratory Biomarkers of Formula (I) activity, safety, and efficacy. Endpoint(s): These biomarkers may include, but are not limited to, FGF23, parathyroid hormone, calcitriol, FGF19, and tumor-derived exosomes
   Evaluate concordance between tissue-based next-generation sequencing (NGS) results and the CTA for detection of FGFR3 gene alterations in participants enrolled in Phase 2, Cohorts 1 and 2
   Estimate OS in Cohorts 1 and 2 in Phase 2

### Efficacy Assessments:

Baseline tumor assessment is completed within 28 days prior to the first dose of study drug. It is recommended to perform CT of the chest, abdomen, pelvic cavity, and any other location where disease is present at baseline; additional tumor imaging evaluation must be performed if clinically indicated or if suspicious lesions are identified in other areas. Baseline brain MRI is not required unless there is a prior history of brain metastases or neurologic symptoms suggestive of new brain metastases. The preferred tumor imaging assessment modality is CT with IV contrast; however, non-contrast CT of the chest and MRI of the abdomen and pelvis are permitted if IV contrast is clinically contraindicated. Baseline and postbaseline efficacy assessments are performed using the same imaging method and, as far as possible, by the same investigator (RECIST v1.1).

Tumor imaging assessment should be performed every 8 weeks (±7 days) until Cycle 13, Day 1, and then every 12 weeks (±7 days) thereafter, regardless of study treatment status, until disease progression, death, unacceptable toxicity, withdrawal of participant's consent for treatment, or withdrawal from the study, whichever occurs first. The timing of tumor imaging assessments is calculated based on the date of the first dose of study drug (i.e., Cycle 1, Day 1). Participants who discontinue study drug for reasons other than disease progression or death should continue imaging assessments per the protocol-defined schedule until disease progression, death, withdrawal of participant's consent for treatment, or withdrawal from the study. If an imaging result shows CR or PR for the first time, it is necessary to repeat the tumor imaging to confirm the response; confirmatory tumor imaging must be performed at least 4 weeks after the prior imaging assessment. Further details are provided in an Imaging Manual.

For participants enrolled in Phase 2, Cohorts 1 and 2 only, digital images of scans performed are transmitted from the sites electronically to the sponsor-designated central radiology vendor for “collect and hold”.

Efficacy assessments include ORR, DCR, DOR, TTR, and PFS (RECIST v1.1).

### Response Evaluation Criteria in Solid Tumors Guidelines:

The RECIST v1.1 guideline is presented below. See Eisenhauser E A, Therasse P, Bogaerts J, et al. New response evaluation criteria in solid tumours: Revised RECIST guideline (version 1.1). Eur J Cancer. 2009; 45:228-47.

### Definitions

At baseline, tumor lesions/lymph nodes will be categorized measurable or non-measurable as follows:

### Measurable

Tumor lesions: Must be accurately measured in at least 1 dimension (longest diameter in the plane of measurement is to be recorded) with a minimum size of:
10 mm by CT scan (CT scan slice thickness no greater than 5 mm).
10 mm caliper measurement by clinical exam (lesions which cannot be accurately measured with calipers should be recorded as non-measurable).
20 mm by chest X-ray.

Malignant lymph nodes: To be considered pathologically enlarged and measurable, a lymph node must be ≥15 mm in short axis when assessed by CT scan (CT scan slice thickness recommended to be no greater than 5 mm). At baseline and in follow-up, only the short axis will be measured and followed.

### Non-Measurable

All other lesions, including small lesions (longest diameter <10 mm or pathological lymph nodes with ≥10 to <15 mm short axis) as well as truly non-measurable lesions. Lesions considered truly non-measurable include: leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

### Special Considerations Regarding Lesion Measurability

### Bone Lesions:

Bone scan, positron emission tomography (PET) scan or plain films are not considered adequate imaging techniques to measure bone lesions. However, these techniques can be used to confirm the presence or disappearance of bone lesions.
Lytic bone lesions or mixed lytic-blastic lesions, with identifiable soft tissue components, that can be evaluated by cross sectional imaging techniques such as CT or MRI can be considered as measurable lesions if the soft tissue component meets the definition of measurability described above.
Blastic bone lesions are non-measurable.

### Cystic Lesions:

Lesions that meet the criteria for radiographically defined simple cysts should not be considered as malignant lesions (neither measurable nor non-measurable) since they are, by definition, simple cysts.
‘Cystic lesions’ thought to represent cystic metastases can be considered as measurable lesions, if they meet the definition of measurability described above. However, if noncystic lesions are present in the same participant, these are preferred for selection as target lesions.
   Lesions with Prior Local Treatment:
Tumor lesions situated in a previously irradiated area, or in an area subjected to other locoregional therapy, are usually not considered measurable unless there has been demonstrated progression in the lesion. Study protocols should detail the conditions under which such lesions would be considered measurable.

### Method of Assessment

### Measurement of Lesions

All measurements should be recorded in metric notation, using calipers if clinically assessed. All baseline evaluations should be performed as close as possible to the treatment start and never more than 4 weeks before the beginning of the treatment.

The same method of assessment and the same technique should be used to characterize each identified and reported lesion at baseline and during follow-up. Imaging based evaluation should always be done rather than clinical examination unless the lesion(s) being followed cannot be imaged but are assessable by clinical exam.
Clinical lesions: Clinical lesions are only considered measurable when they are superficial and >10 mm diameter as assessed using calipers (eg, skin nodules). For the case of skin lesions, documentation by color photography including a ruler to estimate the size of the lesion is suggested. As noted above, when lesions can be evaluated by both clinical exam and imaging, imaging evaluation should be undertaken since it is more objective and may also be reviewed at the end of the study.
Chest X-ray: Chest CT is preferred over chest X-ray, particularly when progression is an important endpoint, since CT is more sensitive than X-ray, particularly in identifying new lesions. However, lesions on chest X-ray may be considered measurable if they are clearly defined and surrounded by aerated lung.
CT, MRI: CT is the best currently available and reproducible method to measure lesions selected for response assessment. This guideline has defined measurability of lesions on CT scan based on the assumption that CT slice thickness is 5 mm or less. When CT scans have slice thickness greater than 5 mm, the minimum size for a measurable lesion should be twice the slice thickness. MRI is also acceptable in certain situations (eg, for body scans).
Ultrasound: Ultrasound is not useful in assessment of lesion size and should not be used as a method of measurement. Ultrasound examinations cannot be reproduced in their entirety for independent review at a later date and, because they are operator dependent, it cannot be guaranteed that the same technique and measurements will be taken from 1 assessment to the next. If new lesions are identified by ultrasound in the course of the study, confirmation by CT or MRI is advised. If there is concern about radiation exposure at CT, MRI may be used instead of CT in selected instances.
Endoscopy, laparoscopy: The utilization of these techniques for objective tumor evaluation is not advised. However, they can be useful to confirm complete pathological response when biopsies are obtained or to determine relapse in studies where recurrence following CR or surgical resection is an endpoint.
Tumor markers: Tumor markers alone cannot be used to assess objective tumor response. If markers are initially above the upper normal limit, however, they must normalize for a participant to be considered in CR. Because tumor markers are disease specific, instructions for their measurement should be incorporated into protocols on a disease specific basis. Specific guidelines for both CA-125 response (in recurrent ovarian cancer) and prostate-specific antigen response (in recurrent prostate cancer), have been published. In addition, the Gynecologic Cancer Intergroup has developed CA125 progression criteria which are to be integrated with objective tumor assessment for use in first-line studies in ovarian cancer.
Cytology, histology: These techniques can be used to differentiate between PR and CR in rare cases if required by protocol (for example, residual lesions in tumor types such as germ cell tumors, where known residual benign tumors can remain). When effusions are known to be a potential adverse effect of treatment (eg, with certain taxane compounds or angiogenesis inhibitors), the cytological confirmation of the neoplastic origin of any effusion that appears or worsens during treatment can be considered if the measurable tumor has met criteria for response or SD in order to differentiate between response (or SD) and PD.

### Response Criteria

### Evaluation of Target Lesions

CR: Disappearance of all target lesions. Any pathological lymph nodes (whether target or nontarget) must have reduction in short axis to <10 mm.
PR: At least a 30% decrease in the sum of diameters of target lesions, taking as reference the baseline sum diameters.
PD: At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm (note: the appearance of 1 or more new lesions is also considered progression).
SD: Neither sufficient shrinkage to qualify for PR nor sufficient increase to qualify for PD, taking as reference the smallest sum of diameters while on study.
Lymph nodes: Lymph nodes identified as target lesions should always have the actual short axis measurement recorded (measured in the same anatomical plane as the baseline examination), even if the nodes regress to below 10 mm on study. This means that when lymph nodes are included as target lesions, the ‘sum’ of lesions may not be zero even if CR criteria are met, since a normal lymph node is defined as having a short axis of <10 mm. Case report forms or other data collection methods may therefore be designed to have target nodal lesions recorded in a separate section where, in order to qualify for CR, each node must achieve a short axis <10 mm. For PR, SD and PD, the actual short axis measurement of the nodes is to be included in the sum of target lesions.
Target lesions that become ‘too small to measure’: While on study, all lesions (nodal and non-nodal) recorded at baseline should have their actual measurements recorded at each subsequent evaluation, even when very small (eg, 2 mm). However, sometimes lesions or lymph nodes which are recorded as target lesions at baseline become so faint on CT scan that the radiologist may not feel comfortable assigning an exact measure and may report them as being ‘too small to measure’. When this occurs it is important that a value be recorded on the case report form. If the lesion has likely disappeared, the measurement should be recorded as 0 mm. If the lesion is believed to be present and is faintly seen but too small to measure, a default value of 5 mm should be assigned (Note: It is less likely that this rule will be used for lymph nodes since they usually have a definable size when normal and are frequently surrounded by fat such as in the retroperitoneum; however, if a lymph node is believed to be present and is faintly seen but too small to measure, a default value of 5 mm should be assigned in this circumstance as well). This default value is derived from the 5 mm CT slice thickness (but should not be changed with varying CT slice thickness). The measurement of these lesions is potentially non-reproducible, therefore providing this default value will prevent false responses or progressions based upon measurement error. To reiterate, however, if the radiologist is able to provide an actual measure, that should be recorded, even if it is below 5 mm.
Lesions that split or coalesce on treatment: When non-nodal lesions ‘fragment’, the longest diameters of the fragmented portions should be added together to calculate the target lesion sum. Similarly, as lesions coalesce, a plane between them may be maintained that would aid in obtaining maximal diameter measurements of each individual lesion. If the lesions have truly coalesced such that they are no longer separable, the vector of the longest diameter in this instance should be the maximal longest diameter for the ‘coalesced lesion’.

### Evaluation of Nontarget Lesions

While some nontarget lesions may actually be measurable, they need not be measured and instead should be assessed only qualitatively at the time points specified in the protocol.
CR: Disappearance of all nontarget lesions and normalization of tumor marker level. All lymph nodes must be non-pathological in size (<10 mm short axis).
Non-CR/Non-PD: Persistence of 1 or more nontarget lesion(s) and/or maintenance of tumor marker level above the normal limits.
PD: Unequivocal progression (see comments below) of existing nontarget lesions (note: the appearance of 1 or more new lesions is also considered progression).
When the participant also has measurable disease: In this setting, to achieve ‘unequivocal progression’ on the basis of the nontarget disease, there must be an overall level of substantial worsening in nontarget disease such that, even in presence of SD or PR in target disease, the overall tumor burden has increased sufficiently to merit discontinuation of therapy (see further details below). A modest ‘increase’ in the size of 1 or more nontarget lesions is usually not sufficient to quality for unequivocal progression status. The designation of overall progression solely on the basis of change in nontarget disease in the face of SD or PR of target disease will therefore be extremely rare.
When the participant has only non-measurable disease: This circumstance arises in some Phase 3 studies when it is not a criterion of study entry to have measurable disease. The same general concepts apply here as noted above, however, in this instance there is no measurable disease assessment to factor into the interpretation of an increase in non-measurable disease burden. Because worsening in nontarget disease cannot be easily quantified (by definition: if all lesions are truly non-measurable) a useful test that can be applied when assessing participants for unequivocal progression is to consider if the increase in overall disease burden based on the change in non-measurable disease is comparable in magnitude to the increase that would be required to declare PD for measurable disease: ie, an increase in tumor burden representing an additional 73% increase in ‘volume’ (which is equivalent to a 20% increase diameter in a measurable lesion). Examples include an increase in a pleural effusion from ‘trace’ to ‘large’, an increase in lymphangitic disease from localized to widespread, or may be described in protocols as ‘sufficient to require a change in therapy’. If ‘unequivocal progression’ is seen, the participant should be considered to have had overall PD at that point. While it would be ideal to have objective criteria to apply to non-measurable disease, the very nature of that disease makes it impossible to do so, therefore the increase must be substantial.

### New Lesions

The appearance of new malignant lesions denotes disease progression; therefore, some comments on detection of new lesions are important. There are no specific criteria for the identification of new radiographic lesions; however, the finding of a new lesion should be unequivocal: i.e., not attributable to differences in scanning technique, change in imaging modality or findings thought to represent something other than tumor (for example, some ‘new’ bone lesions may be simply healing or flare of pre-existing lesions). This is particularly important when the participant's baseline lesions show partial or CR. For example, necrosis of a liver lesion may be reported on a CT scan report as a ‘new’ cystic lesion, which it is not.

A lesion identified on a follow-up study in an anatomical location that was not scanned at baseline is considered a new lesion and will indicate disease progression. An example of this is the participant who has visceral disease at baseline and while on study has a CT or MRI brain ordered which reveals metastases. The participant's brain metastases are considered to be evidence of PD even if he/she did not have brain imaging at baseline.

If a new lesion is equivocal, for example because of its small size, continued therapy and follow-up evaluation will clarify if it represents truly new disease. If repeat scans confirm there is definitely a new lesion, then progression should be declared using the date of the initial scan.

While fluorodeoxyglucose (FDG)-PET response assessments need additional study, it is sometimes reasonable to incorporate the use of FDG-PET scanning to complement CT scanning in assessment of progression (particularly possible ‘new’ disease). New lesions on the basis of FDG-PET imaging can be identified according to the following algorithm:
a. Negative FDG-PET at baseline, with a positive FDG-PET at follow-up is a sign of PD based on a new lesion (a ‘positive’ FDG-PET scan lesion means 1 which is FDG avid with an uptake greater than twice that of the surrounding tissue on the attenuation corrected image).
b. No FDG-PET at baseline and a positive FDG-PET at follow-up:
   i. If the positive FDG-PET at follow-up corresponds to a new site of disease confirmed by CT, this is PD. If the positive FDG-PET at follow-up is not confirmed as a new site of disease on CT, additional follow-up CT scans are needed to determine if there is truly progression occurring at that site (if so, the date of PD will be the date of the initial abnormal FDG-PET scan).
   ii. If the positive FDG-PET at follow-up corresponds to a pre-existing site of disease on CT that is not progressing on the basis of the anatomic images, this is not PD.

### Evaluation of Best Overall Response

The BOR is the best response recorded from the start of the study treatment until the EOT taking into account any requirement for confirmation. On occasion a response may not be documented until after the end of therapy so protocols should be clear if post-treatment assessments are to be considered in determination of BOR. Protocols must specify how any new therapy introduced before progression will affect best response designation. The participant's BOR assignment depends on the findings of both target and nontarget disease and also takes into consideration the appearance of new lesions. Furthermore, depending on the nature of the study and the protocol requirements, it may also require confirmatory measurement. Specifically, in non-randomized studies where response is the primary endpoint, confirmation of PR or CR is needed to deem either one the ‘BOR’.

The BOR is determined once all the data for the participant is known. Best response determination in studies where confirmation of complete or PR is not required: best response in these studies is defined as the best response across all time points (eg, a participant who has SD at first assessment, PR at second assessment, and PD on last assessment has a BOR of PR). When SD is believed to be best response, it must also meet the protocol-specified minimum time from baseline. If the minimum time is not met when SD is otherwise the best time point response, the participant's best response depends on the subsequent assessments. For example, a participant who has SD at first assessment, PD at second and does not meet minimum duration for SD, will have a best response of PD. The same participant lost to follow-up after the first SD assessment would be considered inevaluable.

Time Point Response: Participants
with Target (±Nontarget) Disease

| | | | Overall |
|---|---|---|---|
| Target Lesions | Nontarget Lesions | New Lesions | Response |
| CR | CR | No | CR |
| CR | Non-CR/non-PD | No | PR |
| CR | Not evaluated | No | PR |
| PR | Non-PD or not all | No | PR |
| | evaluated | | |
| SD | Non-PD or not all | No | SD |
| | evaluated | | |
| Not all evaluated | Non-PD | No | NE |
| PD | Any | Yes or No | PD |
| Any | PD | Yes or No | PD |
| Any | Any | Yes | PD |

Abbreviations: CR = complete response, NE = not evaluable, PD = progressive disease, PR = partial response, SD = stable disease.

Time Point Response: Participants with Nontarget Disease Only

| Nontarget Lesions | New Lesions | Overall Response |
|---|---|---|
| CR | No | CR |
| Non-CR/non-PD | No | Non-CR/non-PR^{a} |
| Not all evaluated | No | NE |
| Unequivocal PD | Yes or No | PD |
| Any | Yes | PD |

^{a}‘Non-CR/non-PD’ is preferred over ‘stable disease’ for nontarget disease since SD is increasingly used as endpoint for assessment of efficacy in some studies so to assign this category when no lesions can be measured is not advised.
Abbreviations: CR = complete response, NE = not evaluable, PD = progressive disease.

Best response determination in studies where confirmation of complete or PR is required: CR or PR may be claimed only if the criteria for each are met at a subsequent time point as specified in the protocol (generally 4 weeks later). In this circumstance, the BOR can be interpreted as in the table below.

Best Overall Response when Confirmation of CR and PR Required

| Overall | Overall | |
|---|---|---|
| Response, | Response, | |
| First | Subsequent | |
| Time Point | Time Point | Best Overall Response |
| CR | CR | CR |
| CR | PR | SD, PD, or PR^{a} |
| CR | SD | SD provided minimum criteria for SD |
| | | duration met, otherwise PD |
| CR | PD | SD provided minimum criteria for SD |
| | | duration met, otherwise PD |
| CR | NE | SD provided minimum criteria for SD |
| | | duration met, otherwise NE |
| PR | CR | PR |
| PR | PR | PR |
| PR | SD | SD |
| PR | PD | SD provided minimum criteria for SD |
| | | duration met, otherwise PD |
| PR | NE | SD provided minimum criteria for SD |
| | | duration met, otherwise NE |
| NE | NE | NE |

^{a}If a CR is truly met at first time point, then any disease seen at a subsequent time point, even disease meeting PR criteria relative to baseline, makes the disease PD at that point (since disease must have reappeared after CR). Best response would depend on whether minimum duration for SD was met. However, sometimes ‘CR’ may be claimed when subsequent scans suggest small lesions were likely still present and in fact the participant had PR, not CR at the first time point. Under these circumstances, the original CR should be changed to PR and the best response is PR.
Abbreviations: CR = complete response, NE = not evaluable, PD = progressive disease, PR = partial response, SD = stable disease.

When nodal disease is included in the sum of target lesions and the nodes decrease to ‘normal’ size (<10 mm), they may still have a measurement reported on scans. This measurement should be recorded even though the nodes are normal in order not to overstate progression should it be based on increase in size of the nodes. As noted earlier, this means that participants with CR may not have a total sum of ‘zero’ on the eCRF.

In studies where confirmation of response is required, repeated ‘NE’ time point assessments may complicate best response determination. The analysis plan for the study must address how missing data/assessments will be addressed in determination of response and progression. For example, in most studies it is reasonable to consider a participant with time point responses of PR-NE-PR as a confirmed response.

Participants with a global deterioration of health status requiring discontinuation of treatment without objective evidence of disease progression at that time should be reported as ‘symptomatic deterioration’. Every effort should be made to document objective progression even after discontinuation of treatment. Symptomatic deterioration is not a descriptor of an objective response: it is a reason for stopping study drug. The objective response status of such participants is to be determined by evaluation of target and nontarget disease as shown in the tables herein.

Conditions that define ‘early progression, early death and inevaluability’ are study-specific and should be clearly described in each protocol (depending on treatment duration, treatment periodicity).

In some circumstances it may be difficult to distinguish residual disease from normal tissue. When the evaluation of CR depends upon this determination, it is recommended that the residual lesion be investigated (fine needle aspirate/biopsy) before assigning a status of CR. FDG-PET may be used to upgrade a response to a CR in a manner similar to a biopsy in cases where a residual radiographic abnormality is thought to represent fibrosis or scarring. The use of FDG-PET in this circumstance should be prospectively described in the protocol and supported by disease specific medical literature for the indication. However, it must be acknowledged that both approaches may lead to false positive CR due to limitations of FDG-PET and biopsy resolution/sensitivity.

For equivocal findings of progression (eg, very small and uncertain new lesions; cystic changes or necrosis in existing lesions), treatment may continue until the next scheduled assessment. If at the next scheduled assessment, progression is confirmed, the date of progression should be the earlier date when progression was suspected.

### Confirmatory Measurement and Duration of Response

### Confirmation

In non-randomized studies where response is the primary endpoint, confirmation of PR and CR is required to ensure responses identified are not the result of measurement error. This also permits appropriate interpretation of results in the context of historical data where response has traditionally required confirmation in such studies. However, in all other circumstances, i.e., in randomized studies (Phase 2 or 3) or studies where SD or progression are the primary endpoints, confirmation of response is not required since it will not add value to the interpretation of study results. However, elimination of the requirement for response confirmation may increase the importance of central review to protect against bias, in particular in studies which are not blinded.

In the case of SD, measurements must have met the SD criteria at least once after study entry at a minimum interval (in general not less than 6 to 8 weeks) that is defined in the study protocol.

### Duration of Overall Response

The duration of overall response is measured from the time measurement criteria are first met for CR/PR (whichever is first recorded) until the first date that recurrent or progressive disease is objectively documented (taking as reference for PD the smallest measurements recorded on study).

The duration of overall CR is measured from the time measurement criteria are first met for CR until the first date that recurrent disease is objectively documented.

### Duration of Stable Disease

SD is measured from the start of the treatment (in randomized studies, from date of randomization) until the criteria for progression are met, taking as reference the smallest sum on study (if the baseline sum is the smallest, this is the reference for calculation of PD).

The clinical relevance of the duration of SD varies in different studies and diseases. If the proportion of participants achieving SD for a minimum period of time is an endpoint of importance in a particular study, the protocol should specify the minimal time interval required between 2 measurements for determination of SD.

Note: The DOR and SD as well as the PFS are influenced by the frequency of follow-up after baseline evaluation. The frequency should take into account many parameters including disease types and stages, treatment periodicity and standard practice. However, these limitations of the precision of the measured endpoint should be taken into account if comparisons between studies are to be made.

### Pharmacokinetic Assessments

Plasma samples are collected for the measurement of plasma concentrations of Formula (I). Samples may be collected at additional timepoints during the study, if warranted. Samples collected for analyses of Formula (I) plasma concentrations may also be used to evaluate safety or efficacy aspects related to concerns arising during or after the study. The actual date and time (24-hour clock time) of each sample is recorded. The testing of PK samples is performed by a designated bioanalytical contract research organization with a validated method.

| Phase | Study Visit | Timing of Sample (2 mL) |
|---|---|---|
| Phase 1 (Parts A and B) | Cycle 1, Day 1 | Predose |
| | | 30 minutes postdose (+10 min) |
| | | 1 hour postdose (±10 min) |
| | | 2 hours postdose (±10 min) |
| | | 4 hours postdose (±10 min) |
| | | 8 hours postdose (±10 min) |
| | | 24 hours postdose (±1 hour) |
| | Cycle 1, Day 15 | Predose |
| | | 30 minutes postdose (+10 min) |
| | | 1 hour postdose (±10 min) |
| | | 2 hours postdose (±10 min) |
| | | 4 hours postdose (±10 min) |
| | | 8 hours postdose (±10 min) |
| | | 24 hours postdose (±1 hour) |
| | Cycle 2, Day 1 | Predose |
| | Cycle 2, Day 15 | Predose |
| | Cycle 3, Day 1 | Predose |
| | Unscheduled visit due to an AE^{a} | Anytime during visit |
| Phase 2 | Cycle 1, Day 1 | 1 hour postdose (±10 min) |
| | | 2 hours postdose (±10 min) |
| | | 4 hours postdose (±10 min) |
| | Cycle 2, Day 1 | Predose |
| | | 1 hour postdose (±10 min) |
| | | 2 hours postdose (±10 min) |
| | | 4 hours postdose (±10 min) |
| | Cycle 2, Day 15 | Predose |
| | Cycle 3, Day 1 | Predose |
| | Unscheduled visit due to an AE^{a} | Anytime during visit |

### Pharmacodynamic Assessments

Blood samples are collected for the measurement of circulating biomarkers, including but not limited to FGF23, parathyroid hormone, calcitriol, FGF19, and tumor-derived exosomes. In pediatric participants (12 to 21 years of age), serum Collagen Type X (CXM) may also be evaluated.

Urine samples are collected for assessing secreted biomarkers for FGFR3 inhibition. These markers include, but will not be limited to, matrix metalloproteinase-1, matrix metalloproteinase-10, and fibroblast growth factor binding protein 1.

Further assessments are performed, at the discretion of the sponsor, using excess biomarker or PK samples. Analyses are conducted by a sponsor-designated central laboratory.

| | | Timing of Sample |
|---|---|---|
| Phase | Study Visit | (20 mL) |
| Phase 1 (Parts A and B) and | Cycle 1, Day 1 | Predose |
| Phase 2 | Cycle 1, Day 15 | Predose |
| | Cycle 2, Day 1 | Predose |

Safety Safety is assessed by close monitoring and timely assessment of AEs, Outcome laboratory parameters (hematology and clinical chemistry), vital signs Measures: (blood pressure and heart rate), participant's medical condition (physical examination including weight), ophthalmological examination, and general wellbeing and activities of daily life (ECOG PS).

1. A method of treating cancer in a patient in need thereof, comprising administering to the patient a compound of Formula (I), 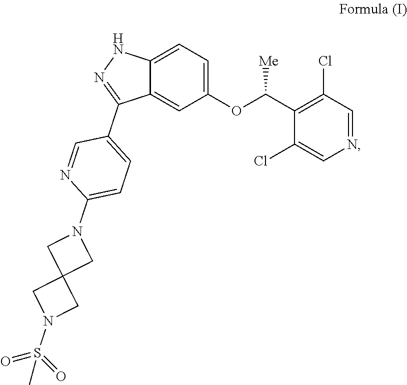 [SMILES: C[C@@H](Oc1ccc2[nH]nc(-c3ccc(N4CC5(C4)CN(S(C)(=O)=O)C5)nc3)c2c1)c1c(Cl)cncc1Cl]
or a pharmaceutically acceptable salt thereof.

2. The method of claim 1, wherein the cancer has an activating FGFR3 gene alteration.

3. The method of claim 2, wherein the cancer is urothelial cancer, breast cancer, endometrial cancer, lung cancer, ovarian cancer, or bladder cancer.

4.-10. (canceled)

11. The method of claim 1, wherein the cancer is a locally advanced solid tumor.

12. The method of claim 1, wherein the cancer is a metastatic solid tumor.

13. The method of claim 1, wherein the activating FGFR3 gene alteration is a mutation.

14. The method of claim 13, wherein the mutation is or comprises one or more of
FGFR3 p.S84L;
FGFR3 p.G380R;
FGFR3 p.R621H;
FGFR3 p.R248C;
FGFR3 p.G380E;
FGFR3 p.K650E;
FGFR3 p.S249C;
FGFR3 p.A391V;
FGFR3 p.K650M;
FGFR3 p.P250R;
FGFR3 p.A391E;
FGFR3 p.K650T;
FGFR3 p.T264M;
FGFR3 p.M528I;
FGFR3 p.K650N;
FGFR3 p.G370C;
FGFR3 p.N540D;
FGFR3 p.R669Q;
FGFR3 p.S371C;
FGFR3 p.N540S;
FGFR3 p.G697C;
FGFR3 p.Y373C; or
FGFR3 p.N540K.

15. The method of claim 13, wherein the mutation is or comprises one or more of
FGFR3 p.V553M;
FGFR3 p.V555M; or
FGFR3 p.V555L.

16. The method of claim 2, wherein the activating FGFR3 gene alteration is a fusion.

17. The method of claim 16, wherein the fusion is an FGFR3 rearrangements with an intact FGFR3 kinase domain and:
Breakpoint in intron 17 or exon 18 of FGFR3 and a known partner gene (e.g., TACC3, BAIAP2L1);
Breakpoint in intron 17 or exon 18 of FGFR3 and an in-frame novel partner gene; or
Breakpoint in intron 17 or exon 18 of FGFR3 and an intra-genic region or out-of-frame partner gene.

18. The method of claim 1, wherein the patient is administered a compound of Formula (I).

19. The method of claim 1, wherein the patient is administered a pharmaceutically acceptable salt of a compound of Formula (I).

20. The method of claim 19, wherein the pharmaceutically acceptable salt of a compound of Formula (I) is the besylate salt.

21. The method of claim 1, wherein the patient is administered the compound of Formula (I), or a pharmaceutically acceptable salt of a compound of Formula (I) (on a Formula (I) basis), in an amount of 10 mg-120 mg per day of.

22.-27. (canceled)

28. The method of claim 21, wherein the compound of Formula (I), or pharmaceutically acceptable salt of a compound of Formula (I), is administered orally.

29. The method of claim 1, wherein the compound of Formula (I), or pharmaceutically acceptable salt of a compound of Formula (I), is administered for at least 28 days.

30. The method of claim 29, wherein the compound of Formula (I), or pharmaceutically acceptable salt of a compound of Formula (I), is administered for 28 days.

31. The method of claim 1, wherein the cancer exhibits a complete response (CR) or a partial response (PR), as evaluated by the Response Evaluation Criteria in Solid Tumors (RECIST) v1.1 criteria, to the administration of the compound of Formula (I), or pharmaceutically acceptable salt of a compound of Formula (I).

32. The method of claim 31, wherein the cancer exhibits a complete response (CR), as evaluated by the Response Evaluation Criteria in Solid Tumors (RECIST) v1.1 criteria, to the administration of the compound of Formula (I), or pharmaceutically acceptable salt of a compound of Formula (I).

33. The method of claim 31, wherein the cancer exhibits a partial response (PR), as evaluated by the Response Evaluation Criteria in Solid Tumors (RECIST) v1.1 criteria, to the administration of the compound of Formula (I), or pharmaceutically acceptable salt of a compound of Formula (I).

34.-66. (canceled)
